(19) United States
(12) Patent Application Publication
(10) Pub. No.: US 2026/0253218 A1
(43) Pub. Date: Aug. 27, 2026

(54) SYSTEM

(71) Applicant: SoftBank Group Corp., Tokyo (JP)

(72) Inventor: Jiro YABUNAKA, Tokyo (JP)

(73) Assignee: SoftBank Group Corp., Tokyo (JP)

(21) Appl. No.: 19/542,696

(22) Filed: Feb. 18, 2026

(30) Foreign Application Priority Data
Feb. 21, 2025 (JP) 2025-026980

(51) Int. Cl. G06T 7/00; G06T 7/11; G06T 17/20; G06V 10/764; G06V 10/82; G06V 40/16; G10L 25/63; G16H 40/67

(52) U.S. Cl. CPC G06T 7/0012; G06T 7/11; G06T 17/20; G06V 10/764; G06V 10/82; G16H 40/67; G06T 2207/10024; G06T 2207/20081; G06T 2207/20084; G06T 2207/30036; G06T 2210/41; G06V 40/174; G06V 2201/03; G10L 25/63

(57) ABSTRACT

The system according to the embodiment comprises a camera unit, an analysis unit, a specifying unit, a generation unit, a sharing unit, and an analysis unit. The camera unit captures images of the inside of the mouth. The analysis unit analyzes images captured by the camera unit using AI. The specifying unit identifies areas requiring treatment based on results analyzed by the analysis unit. The generation unit generates images of the areas requiring treatment identified by the specifying unit. The sharing unit shares images generated by the generation unit or past symptoms and treatment methods between a patient and a doctor. The analysis unit converts oral data of a plurality of patients into big data and analyzes it using generative AI.

### CROSS-REFERENCE TO RELATED APPLICATION

The present application claims priority to and incorporates by reference the entire contents of Japanese Patent Application No. 2025-026980 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, there has been a problem in that the process of identifying areas requiring treatment inside the mouth and sharing past symptoms and treatment methods between a patient and a doctor has not been performed efficiently.

### SUMMARY OF THE INVENTION

The system according to the embodiment comprises a camera unit, an analysis unit, a specifying unit, a generation unit, a sharing unit, and an analysis unit. The camera unit captures images of the inside of the mouth. The analysis unit analyzes images captured by the camera unit using AI. The specifying unit identifies areas requiring treatment based on results analyzed by the analysis unit. The generation unit generates images of the areas requiring treatment identified by the specifying unit. The sharing unit shares images generated by the generation unit or past symptoms and treatment methods between a patient and a doctor. The analysis unit converts oral data of a plurality of patients into big data and analyzes it using generative AI.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

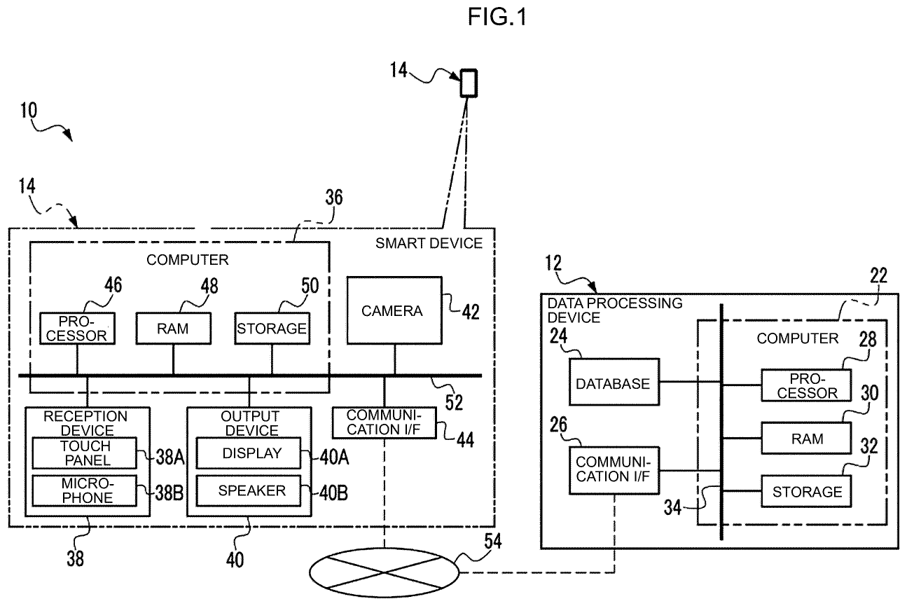

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

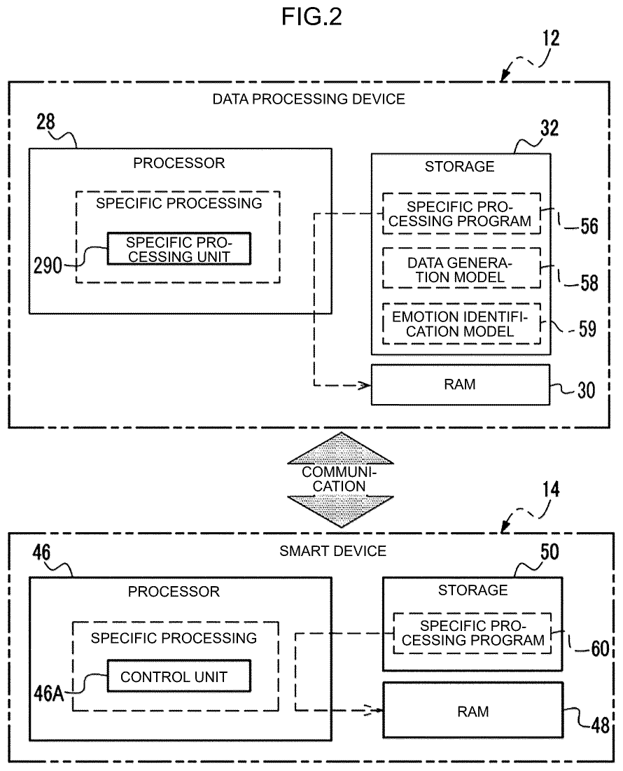

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The system according to the embodiment of the present invention is a system that links daily oral conditions with a healthcare application using an electric toothbrush equipped with an AI camera. This system captures images of the inside of the mouth using an electric toothbrush with an AI camera, analyzes them with AI to identify areas requiring treatment, generates images of the identified areas, and shares them along with past symptoms and treatment methods between the patient and the doctor. This information is provided through a healthcare application to encourage medical action by the patient. Furthermore, oral data from tens of thousands to hundreds of millions of patients is converted into big data and analyzed by generative AI, thereby contributing to the advancement of future medical care. For example, the system captures images of the inside of the mouth using an electric toothbrush equipped with an AI camera. At this time, the camera built into the toothbrush captures detailed images of the inside of the mouth, such as the surface of the teeth and the condition of the gums, in high resolution. This enables detailed recording of daily oral conditions. Next, the captured images are analyzed by AI, which identifies areas requiring treatment based on the images. For example, signs of cavities or periodontal disease can be detected, and areas requiring treatment can be identified, enabling early detection of problems and appropriate treatment. Images of the identified areas requiring treatment are generated and shared between the patient and the doctor along with past symptoms and treatment methods. This information is provided through the healthcare application. For example, when the patient opens the application, images of areas requiring treatment, past symptoms, and treatment methods are displayed, allowing the patient to understand their oral condition and share it with the doctor. Furthermore, oral data from tens of thousands to hundreds of millions of patients is converted into big data and analyzed by generative AI. The generative AI analyzes the collected data to contribute to the advancement of future medical care, such as analyzing common symptoms and treatment methods, developing new treatment methods, and proposing preventive measures, thereby improving the quality of medical care. Through this mechanism, daily oral conditions can be recorded in detail and problems can be detected early. In addition, sharing information between the patient and the doctor enables appropriate treatment, and utilizing big data contributes to the advancement of future medical care. Thus, the system using an electric toothbrush equipped with an AI camera can record daily oral conditions in detail, detect problems early, and provide appropriate treatment. Furthermore, sharing information between the patient and the doctor promotes medical action and contributes to the advancement of medical care. Specifically, the system is composed of multiple hardware and software modules such as a camera unit, analysis unit, specifying unit, generation unit, sharing unit, and analysis unit. The camera unit uses a high-resolution CMOS sensor built into the toothbrush to acquire, for example, 1920×1080 pixel color images and multiple consecutive images (video frames). Examples of input data include RGB image tensors (3×1080×1920) or time-series image arrays (4D tensors for 10 frames). These images are preprocessed, such as noise removal and contrast correction, and then transferred to the analysis unit. The analysis unit uses image recognition models such as convolutional neural networks (CNN) and Vision Transformers to automatically extract features such as tooth surface opacity, discoloration, gum swelling, and bleeding marks. Examples of AI input include the aforementioned image tensors and region-of-interest (ROI) images for each dental arch (e.g., 128×128 pixels). AI output includes abnormal probability maps for each pixel (continuous values from 0.0 to 1.0) or labeled segmentation maps (e.g., 2D arrays) such as “suspected cavity” or “suspected periodontal disease.” For example, output examples include “upper left second molar: cavity suspicion score 0.87” and “lower right canine: no abnormality.” The specifying unit automatically lists areas requiring treatment by threshold judgment (e.g., score of 0.7 or higher) or rule-based logic (priority assignment when multiple areas are detected simultaneously) based on the output of the analysis unit. Output examples of the specifying unit include “list of areas requiring treatment: upper left second molar, lower right first premolar.” The generation unit generates 2D images of areas requiring treatment or creates three-dimensional oral models using 3D reconstruction algorithms (e.g., multi-view stereo method, mesh generation) based on the results of the specifying unit. The generated images are output in various formats, such as color information, highlighted abnormal areas, and simulation images before and after treatment. Output examples include “3D mesh data (obj format)” and “2D images with abnormal areas highlighted in red.” The sharing unit transmits generated images and past treatment history (e.g., time-series database, treatment content text, doctor comments) to the healthcare application using encrypted communication, making them viewable on patient and doctor terminals. Shared information is displayed on the user interface as detailed views for each area, time-series graphs of treatment history, and feedback messages from doctors. Furthermore, the analysis unit accumulates tens of thousands to hundreds of millions of patient data (images, treatment history, lifestyle questionnaires, etc.) in a distributed database and uses generative AI (e.g., large language models and generative image models) to perform case clustering, statistical analysis of treatment effects, and discovery of new treatment patterns. Examples of AI input include “patient images+treatment history+lifestyle vectors,” and output examples include “treatment effect prediction score,” “proposed text for new treatment methods,” and “risk factor ranking.” These outputs are used to support decision-making for treatment policies in medical settings, provide personalized prevention proposals to patients, and share knowledge among medical institutions. As a technical effect, the present invention achieves significant improvements in image analysis accuracy, earlier diagnosis and treatment, efficient data management, and automatic aggregation and development of medical knowledge compared to conventional visual diagnosis and manual recording/sharing by humans. Application fields include daily oral care in general households, telemedicine, diagnostic support in dental clinics, medical big data analysis, and personalized healthcare services.

The electric toothbrush system equipped with an AI camera according to the embodiment comprises a camera unit, an analysis unit, a specifying unit, a generation unit, a sharing unit, and an analysis unit. The camera unit captures images of the inside of the mouth. For example, the camera unit captures detailed images of the inside of the mouth using a camera built into the toothbrush. The camera unit can capture high-resolution images of the surface of the teeth and the condition of the gums, for example. The analysis unit analyzes images captured by the camera unit using AI. For example, the analysis unit uses AI to analyze the captured images and detect signs of cavities or periodontal disease. The analysis unit may analyze images using deep learning or machine learning algorithms. The specifying unit identifies areas requiring treatment based on the results analyzed by the analysis unit. For example, the specifying unit identifies areas requiring treatment for cavities or periodontal disease based on the analysis results. The generation unit generates images of the areas requiring treatment identified by the specifying unit. For example, the generation unit generates 2D images or 3D models of the identified areas requiring treatment. The sharing unit shares images generated by the generation unit, past symptoms, and treatment methods between the patient and the doctor. For example, the sharing unit shares information through a healthcare application. When the patient opens the application, images of areas requiring treatment, past symptoms, and treatment methods are displayed. The analysis unit converts oral data of tens of thousands to hundreds of millions of patients into big data and analyzes it using generative AI. For example, the analysis unit analyzes common symptoms and treatment methods based on the collected data and proposes the development of new treatment methods and preventive measures. Thus, the electric toothbrush system equipped with an AI camera according to the embodiment can record daily oral conditions in detail, detect problems early, and provide appropriate treatment. Furthermore, sharing information between the patient and the doctor promotes medical action and contributes to the advancement of medical care. Specifically, each module of the system, including the camera unit, analysis unit, specifying unit, generation unit, sharing unit, and analysis unit, can be implemented on an embedded system equipped with a dedicated microprocessor, memory, and communication interface. The camera unit is equipped with a high-resolution CMOS sensor (1920×1080 pixels), for example, and can acquire RGB image tensors (3×108×1920) and continuous frames (4D tensors for 10 frames). These images are preprocessed, such as noise removal and contrast correction, and then transferred to the analysis unit. The analysis unit uses image recognition models such as convolutional neural networks (CNN) and Vision Transformers to automatically extract features such as tooth surface opacity, discoloration, gum swelling, and bleeding marks. Examples of AI input include image tensors and ROI images for each dental arch (128×128 pixels). AI output includes abnormal probability maps (continuous values from 0.0 to 1.0), labeled segmentation maps (2D arrays), and, for example, “upper left second molar: cavity suspicion score 0.87” and “lower right canine: no abnormality.” The specifying unit automatically lists areas requiring treatment by threshold judgment (score of 0.7 or higher) or rule-based logic. Output examples of the specifying unit include “list of areas requiring treatment: upper left second molar, lower right first premolar.” The generation unit generates three-dimensional oral models using 2D image extraction algorithms or 3D reconstruction algorithms (multi-view stereo method, mesh generation) based on the results of the specifying unit. The generated images are output in various formats, such as color information, highlighted abnormal areas, and simulation images before and after treatment. Output examples include “3D mesh data (obj format)” and “2D images with abnormal areas highlighted in red.” The sharing unit transmits generated images and treatment history (time-series database, treatment content text, doctor comments) to the healthcare application using encrypted communication, making them viewable on patient and doctor terminals. Shared information is displayed as detailed views for each area, time-series graphs of treatment history, and feedback messages from doctors. The analysis unit accumulates tens of thousands to hundreds of millions of patient data (images, treatment history, lifestyle questionnaires, etc.) in a distributed database and uses generative AI (large language models and generative image models) to perform case clustering, statistical analysis of treatment effects, and discovery of new treatment patterns. Examples of AI input include “patient images+treatment history+lifestyle vectors,” and output examples include “treatment effect prediction score,” “proposed text for new treatment methods,” and “risk factor ranking.” These outputs are used to support decision-making for treatment policies, personalized prevention proposals, and knowledge sharing among medical institutions. As a technical effect, the present invention achieves significant improvements in image analysis accuracy, earlier diagnosis and treatment, efficient data management, and automatic aggregation and development of medical knowledge compared to conventional visual diagnosis and manual recording/sharing by humans. Application fields include daily oral care in general households, telemedicine, diagnostic support in dental clinics, medical big data analysis, and personalized healthcare services.

The camera unit can capture detailed images of the inside of the mouth using a camera built into a toothbrush. For example, the camera unit captures detailed images of the inside of the mouth using a camera built into a toothbrush. Detailed images may include, for example, the surface of the teeth and the condition of the gums, but are not limited to such examples. The camera unit can also use macro photography to capture enlarged images of specific areas. Thus, by using a camera built into a toothbrush, detailed images of the inside of the mouth can be captured. Some or all of the above-described processing in the camera unit may be performed using AI or without using AI. For example, the camera unit may input the captured images to generative AI and have the generative AI perform image analysis. Specifically, the camera unit is equipped with a high-resolution CMOS sensor built into the toothbrush (e.g., 192×1080 pixels, 24-bit color) and can acquire RGB image tensors (3×1080×1920) and continuous frames (4D tensors for 10 frames). The camera unit can perform real-time preprocessing during image acquisition, such as noise removal (e.g., median filter), contrast correction (e.g., histogram equalization), and distortion correction (e.g., lens distortion parameter correction). Examples of input data include “high-resolution images of the entire dental arch,” “macro images of specific tooth areas,” and “time-series image arrays from continuous shooting.” The camera unit can combine an accelerometer and a gyroscope to perform blur correction and automatic angle adjustment during shooting to follow movements inside the user's mouth. Furthermore, the camera unit is equipped with a real-time image analysis function using AI, which extracts tooth contours and automatically crops regions of interest (ROI) from captured images to optimize data transfer efficiency to the analysis unit. Examples of AI input include “noise-removed RGB image tensors” and “ROI images for each dental arch (128×128 pixels).” Examples of AI output include “sharpness scores for each tooth area,” “automatically cropped ROI coordinates,” and “image quality judgment labels.” These outputs are used to improve abnormality detection accuracy in subsequent analysis units, automatically exclude unnecessary images, and display shooting guides on the user interface. As a technical effect, the camera unit significantly improves image acquisition accuracy, sharpness, and area specificity compared to conventional manual shooting or low-resolution cameras, and reduces the computational load of subsequent processing through automatic preprocessing and ROI extraction by AI, thereby improving overall diagnostic accuracy and processing speed. Application fields include daily oral care in general households, diagnostic support in dental clinics, telemedicine, personalized healthcare services, and medical big data collection.

The analysis unit can analyze images captured by AI and detect signs of cavities or periodontal disease. For example, the analysis unit analyzes images captured by AI to detect signs of cavities or periodontal disease. Signs of cavities or periodontal disease may include, for example, discoloration of the tooth surface or swelling of the gums, but are not limited to such examples. The analysis unit can analyze images using deep learning or machine learning algorithms. Thus, by using AI, signs of cavities or periodontal disease can be detected with high accuracy. Some or all of the above-described processing in the analysis unit may be performed using AI or without using AI. For example, the analysis unit may input the captured images to generative AI and have the generative AI perform image analysis. Specifically, the analysis unit can use image recognition models such as convolutional neural networks (CNN) and Vision Transformers to automatically extract various features such as tooth surface opacity, discoloration, gum swelling, bleeding marks, tartar deposition, and misalignment of the dental arch. Examples of AI input include “noise-removed RGB image tensors,” “ROI images for each dental arch,” and “time-series image arrays.” AI output includes abnormal probability maps for each pixel (continuous values from 0.0 to 1.0), labeled segmentation maps (e.g., 2D arrays), and abnormality scores for each area (e.g., “upper left second molar: cavity suspicion score 0.87”). Output examples include “gum area: swelling suspicion score 0.75” and “tooth surface: discoloration suspicion score 0.62.” The analysis unit can automatically list abnormal areas by threshold judgment (e.g., score of 0.7 or higher) or rule-based logic (priority assignment when multiple areas are detected simultaneously). Furthermore, the analysis unit can accumulate abnormality detection results over time, evaluate progression by comparing with past data, and automatically determine trends of expansion or reduction of abnormal areas. For AI model training, loss functions such as cross-entropy and Dice loss are used, and data augmentation (rotation, scaling, noise addition) and transfer learning are utilized to achieve high-precision abnormality detection even in environments with limited data. As a technical effect, the analysis unit significantly improves reproducibility, accuracy, and speed of abnormality detection compared to conventional visual diagnosis by humans or simple image processing, and achieves reduction of false detection rates and earlier diagnosis. Application fields include dental diagnostic support, telemedicine, self-care applications, medical big data analysis, and personalized preventive medicine.

The specifying unit can identify areas requiring treatment based on the analyzed results. For example, the specifying unit identifies areas requiring treatment based on the results analyzed by the analysis unit. Areas requiring treatment may include, for example, areas of cavities or periodontal disease, but are not limited to such examples. The specifying unit can identify areas requiring treatment for cavities or periodontal disease based on the analysis results. Thus, by identifying areas requiring treatment based on the analysis results, problems can be detected early. Some or all of the above-described processing in the specifying unit may be performed using AI or without using AI. For example, the specifying unit may input the analysis results to generative AI and have the generative AI perform identification of areas requiring treatment. Specifically, the specifying unit receives abnormal probability maps and labeled segmentation maps output from the analysis unit as input, and automatically generates a list of areas requiring treatment for each area by performing threshold judgment (e.g., score of 0.7 or higher) or rule-based prioritization (e.g., sorting by severity when multiple areas are detected simultaneously). Examples of input data include “abnormal probability map (2D array),” “list of abnormality scores for each area,” and “past abnormality history data.” Output examples of the specifying unit include “list of areas requiring treatment: upper left second molar, lower right first premolar,” “priority list of treatment areas,” and “treatment recommendation score.” The specifying unit can also use AI models (e.g., decision trees, random forests, rule-based classifiers) to automatically classify severity of abnormal areas and determine treatment priority. Furthermore, the specifying unit can implement individualized identification logic by combining past treatment history and patient lifestyle data (e.g., smoking history, frequency of sugar intake). Examples of AI input include “abnormality score+lifestyle vector” and “past treatment history+current abnormal areas.” Examples of AI output include “individualized list of treatment areas” and “treatment recommendations with risk factors.” These outputs are used for image generation in the subsequent generation unit, treatment proposals to doctors and patients, and support for treatment planning. As a technical effect, the specifying unit achieves objective and highly reproducible identification of treatment areas, and enables standardization, efficiency, and earlier diagnosis compared to conventional subjective identification and manual listing by humans. Application fields include dental diagnostic support, telemedicine, personalized treatment planning, and medical big data analysis.

The generation unit can generate images of the identified areas requiring treatment. For example, the generation unit generates images of areas requiring treatment identified by the specifying unit. Images of areas requiring treatment may include, for example, 2D images or 3D models, but are not limited to such examples. The generation unit can generate 2D images or 3D models of the identified areas requiring treatment. Thus, by generating images of areas requiring treatment, information can be easily shared between the patient and the doctor. Some or all of the above-described processing in the generation unit may be performed using AI or without using AI. For example, the generation unit may input information of the identified areas requiring treatment to generative AI and have the generative AI perform image generation. Specifically, the generation unit receives the list of areas requiring treatment and area coordinate information output from the specifying unit as input, and can automatically generate three-dimensional oral models or images highlighting abnormal areas using 2D image extraction algorithms (e.g., ROI extraction, image cropping) and 3D reconstruction algorithms (e.g., multi-view stereo method, mesh generation, point cloud processing). Examples of input data include “coordinates of areas requiring treatment+original image,” “image arrays from multiple angles,” and “images before and after past treatment.” Output examples of the generation unit include “2D images highlighting abnormal areas in red,” “3D mesh data (obj format),” “simulation images before and after treatment,” and “images highlighting recommended treatment areas.” The generation unit can also use generative AI (e.g., image generation models, 3D generation models) to automatically highlight abnormal areas, simulate treatment effects, and generate predicted images after treatment. Examples of AI input include “coordinates of areas requiring treatment+original image” and “treatment history+current image.” Examples of AI output include “predicted images after treatment” and “images highlighting abnormal areas.” These outputs are used for information sharing in the subsequent sharing unit, support for decision-making on treatment policies by patients and doctors, and explanation of treatment effects. As a technical effect, the generation unit achieves visualization, improved understanding, and efficient explanation of treatment areas through automatic generation of 3D models and simulation images, compared to conventional manual image editing or presentation of only 2D images. Application fields include dental diagnostic support, treatment plan explanation, telemedicine, self-care applications, and medical education.

The sharing unit can share generated images, past symptoms, and treatment methods between the patient and the doctor through a healthcare application. For example, the sharing unit shares images generated by the generation unit, past symptoms, and treatment methods between the patient and the doctor through a healthcare application. The healthcare application may include, for example, data sharing functions and notification functions, but is not limited to such examples. When the patient opens the application, images of areas requiring treatment, past symptoms, and treatment methods are displayed. Thus, by sharing information through the healthcare application, the patient and the doctor can more easily perform appropriate treatment. Some or all of the above-described processing in the sharing unit may be performed using AI or without using AI. For example, the sharing unit may input information of generated images, past symptoms, and treatment methods to generative AI and have the generative AI perform information sharing. Specifically, the sharing unit can securely transmit structured data such as 2D images, 3D models, treatment history data, and doctor comments output from the generation unit to the healthcare application using encrypted communication protocols (e.g., TLS/SSL). Examples of input data include “images of abnormal areas+treatment history text,” “3D model data+doctor comments,” and “time-series graphs of treatment history.” Output examples of the sharing unit include “detailed display screens for each area on the patient terminal,” “time-series graphs of treatment history,” “feedback messages from doctors,” and “treatment recommendation notifications.” The sharing unit can implement various information presentation functions on the user interface, such as detailed display for each area, time-series graph display of treatment history, display of feedback messages from doctors, and automatic distribution of treatment recommendation notifications. Furthermore, the sharing unit can use AI (e.g., large language models) to automatically generate personalized treatment explanation texts and preventive proposal messages for each patient, thereby promoting patient understanding and behavioral change. Examples of AI input include “treatment history+patient attribute vector” and “images of abnormal areas+treatment recommendation content.” Examples of AI output include “personalized treatment explanation text” and “preventive proposal message.” These outputs are used to improve the efficiency of information sharing between patients and doctors, support decision-making on treatment policies, and promote medical action by patients. As a technical effect, the sharing unit significantly improves the accuracy, efficiency, and patient understanding of information transmission compared to conventional paper-based or verbal explanations, through immediate sharing of digital data, time-series management, and automatic generation of personalized explanations. Application fields include telemedicine, dental diagnostic support, self-care applications, knowledge sharing among medical institutions, and personalized healthcare services.

The analysis unit can convert oral data of multiple patients into big data and analyze it using generative AI. For example, the analysis unit converts oral data of tens of thousands to hundreds of millions of patients into big data and analyzes it using generative AI. Big data conversion may include, for example, data collection methods and database construction methods, but is not limited to such examples. Generative AI may analyze data using generative models or data mining techniques, for example. Thus, by analyzing big data, the advancement of medical care can be promoted. Some or all of the above-described processing in the analysis unit may be performed using AI or without using AI. For example, the analysis unit may input the collected data to generative AI and have the generative AI perform data analysis. Specifically, the analysis unit accumulates tens of thousands to hundreds of millions of patient data (e.g., oral images, treatment history, lifestyle questionnaires, age, gender, and other attribute information) in a distributed database (e.g., NoSQL cluster, cloud storage), and uses generative AI (e.g., large language models, generative image models, clustering algorithms) to perform various analyses such as case clustering, statistical analysis of treatment effects, discovery of new treatment patterns, and risk factor ranking. Examples of AI input include “patient images+treatment history+lifestyle vectors,” “time-series treatment effect data,” and “patient attributes +symptom labels.” Examples of AI output include “treatment effect prediction score,” “proposed text for new treatment methods,” “risk factor ranking,” and “case cluster labels.” The analysis unit can use these outputs for supporting decision-making on treatment policies in medical settings, providing personalized prevention proposals to patients, sharing knowledge among medical institutions, and developing new treatment methods through medical big data analysis. Furthermore, in AI model training, the analysis unit combines various learning methods such as supervised learning, unsupervised learning, transfer learning, and self-supervised learning to achieve high-precision analysis that accommodates data diversity and imbalance. Within the AI model, loss functions (e.g., cross-entropy, mean squared error), weight optimization algorithms (e.g., Adam, SGD), data augmentation (e.g., image rotation, noise addition), and anomaly detection algorithms (e.g., autoencoder, anomaly scoring) are utilized. As a technical effect, the analysis unit achieves automatic aggregation of vast amounts of data, extraction of high-dimensional features, discovery of new patterns, and improved accuracy of treatment effect prediction, thereby greatly contributing to the automatic development of medical knowledge and improvement of medical quality compared to conventional manual aggregation or simple statistical analysis by humans. Application fields include medical big data analysis, development of treatment methods, personalized preventive medicine, knowledge sharing among medical institutions, and self-care applications.

The camera unit can estimate the user's emotions and adjust the timing of image capture based on the estimated emotions. For example, the camera unit estimates the user's emotions and adjusts the timing of image capture based on the estimated emotions. User emotions may include, for example, relaxation, tension, or being in a hurry, but are not limited to such examples. For example, if the user is relaxed, the camera unit starts capturing images at a natural timing. If the user is tense, the camera unit may wait until the user relaxes before starting image capture. Furthermore, if the user is in a hurry, the camera unit may perform image capture quickly. Thus, by adjusting the timing of image capture according to the user's emotions, images can be captured at a natural timing. Emotion estimation is realized using an emotion engine or generative AI, for example, with emotion estimation functions. Generative AI may estimate the user's emotions using technologies such as facial expression recognition and voice analysis. Some or all of the above-described processing in the camera unit may be performed using AI or without using AI. For example, the camera unit may input the user's facial expression data to generative AI and have the generative AI perform emotion estimation. Specifically, the camera unit can acquire the user's facial images and voice waveform data in real time and input them as multidimensional tensors (e.g., facial images: 3×224×224, voice spectrogram: 1×128×256) to an emotion estimation AI model. AI models may include convolutional neural networks (CNN) for classifying emotions from facial images, recurrent neural networks (RNN) for estimating emotions from voice features, or multimodal Transformers. Examples of AI input include “smiling facial image+calm voice spectrogram” and “frowning face+tense high-pitched voice.” AI output includes emotion labels such as “relaxed,” “tense,” or “in a hurry” (e.g., outputting probability values for each emotion using softmax, e.g., relaxed 0.82, tense 0.12, in a hurry 0.06) and emotion intensity scores (continuous values from 0.0 to 1.0). Output examples include “relaxation level 0.85” and “tension level 0.65.” The camera unit uses these emotion estimation results to control the timing of image capture, such as “if relaxation level is 0.7 or higher, capture immediately,” “if tension level is 0.5 or higher, wait 5 seconds,” using rule-based control. Furthermore, the camera unit can accumulate the user's past emotional transitions and capture history in a time-series database and implement individually optimized timing control logic (e.g., automatically learning the optimal waiting time for each user). For AI model training, emotion-labeled facial images and voice datasets are used, and cross-entropy loss and data augmentation (expression changes, voice noise addition) are utilized to improve accuracy. As a technical effect, the camera unit achieves automatic image capture at the optimal timing according to the user's psychological state, significantly improving the rate of acquiring images with natural expressions and oral conditions compared to conventional timer-based or manual operation. Application fields include self-care in general households, dental diagnostic support, telemedicine, stress-free oral imaging for children and the elderly, and personalized healthcare services.

The camera unit can add an autofocus function for preferentially capturing specific areas inside the mouth during image capture. For example, the camera unit adds an autofocus function for preferentially capturing specific areas inside the mouth during image capture. The autofocus function may include, for example, automatically focusing on areas suspected of cavities, but is not limited to such examples. The camera unit can also adjust focus to capture the condition of the gums in detail or optimize focus to detect fine scratches on the tooth surface. Thus, by adding an autofocus function, specific areas can be captured in detail. Some or all of the above-described processing in the camera unit may be performed using AI or without using AI. For example, the camera unit may input image data of the target area to generative AI and have the generative AI perform focus adjustment. Specifically, the camera unit can input real-time oral image tensors (e.g., 3×1080×1920) and use AI models for automatic detection of candidate abnormal areas (e.g., suspected cavity regions, gum swelling areas, fine scratches), such as CNN-based object detection networks or semantic segmentation models. Examples of AI input include “high-resolution images of the entire dental arch,” “macro images of specific areas,” and “time-series image arrays from continuous frames.” AI output includes bounding box coordinates of abnormal areas (e.g., ROI coordinates for upper left second molar), recommended focus score for each area (0.0 to 1.0), and automatically generated focus control signals (e.g., lens drive amount, focus motor control value). Output examples include “suspected cavity area: coordinates (320,240)-(400,320), recommended focus 0.92” and “gum area: coordinates (100,500)-(200,600), recommended focus 0.85.” The camera unit uses these outputs to control the autofocus module in real time, automatically focusing on abnormal or attention areas. Furthermore, the camera unit can learn from the user's past capture history and abnormality detection trends to implement individually optimized focus control logic (e.g., prioritizing high-precision imaging of gum areas for specific users). For AI model training, image datasets with annotated abnormal areas are used, and loss functions such as IoU loss and cross-entropy, as well as data augmentation (rotation, scaling, noise addition), are utilized to improve generalization performance. As a technical effect, the camera unit significantly improves the acquisition rate of high-resolution images of abnormal or attention areas, diagnostic accuracy, abnormality detection rate, and reproducibility compared to conventional manual focus or overall imaging. Application fields include dental diagnostic support, self-care applications, telemedicine, personalized healthcare, and medical big data collection.

The camera unit can simultaneously measure the temperature and humidity inside the mouth during image capture and record them as additional information in the image. For example, the camera unit simultaneously measures the temperature and humidity inside the mouth during image capture and records them as additional information in the image. Measurement of temperature and humidity may include, for example, the use of sensors, but is not limited to such examples. The camera unit can measure the temperature inside the mouth during image capture and record it in the image. The camera unit can also measure the humidity inside the mouth during image capture and record it in the image. Furthermore, the camera unit can combine temperature and humidity data to provide detailed information about the oral environment. Thus, by recording temperature and humidity information, detailed information about the oral environment can be provided. Some or all of the above-described processing in the camera unit may be performed using AI or without using AI. For example, the camera unit may input the measured temperature and humidity data to generative AI and have the generative AI perform data analysis. Specifically, the camera unit uses a high-precision temperature sensor (e.g., thermistor, resolution 0.1° C.) and humidity sensor (e.g., capacitive type, resolution 1% RH) built into the toothbrush to acquire environmental data inside the mouth in real time during image acquisition. Examples of input data include “image tensor+temperature value 36.5° C.+humidity value 85%” and “time-series temperature and humidity data during continuous shooting.” The camera unit records these temperature and humidity data as image metadata with timestamps and stores them in the EXIF information of the image file or a dedicated database. Furthermore, the camera unit can use AI models (e.g., multivariate regression models, anomaly detection neural networks) to automatically evaluate the oral environment by combining temperature and humidity data with image features, and detect abnormal values (e.g., abnormally high humidity indicating periodontal disease risk). Examples of AI input include “temperature 36.8° C.+humidity 90%+gum image” and “temperature 34.5° C.+humidity 60%+tooth surface image.” AI output includes labels or scores such as “normal,” “abnormal (high humidity),” and “risk score 0.78.” Output examples include “temperature and humidity abnormality detection: risk 0.82” and “normal range.” These outputs are used for assisting abnormality detection in subsequent analysis units, proposing environmental improvements to users, and generating detailed reports for doctors. As a technical effect, the camera unit significantly improves the accuracy and reliability of oral health assessment by integratively recording and analyzing physical environmental parameters such as temperature and humidity, compared to conventional diagnosis using only images. Application fields include dental diagnostic support, self-care applications, telemedicine, oral environment monitoring, and medical big data analysis.

The camera unit can estimate the user's emotions and determine the priority of areas to be captured based on the estimated emotions. For example, the camera unit estimates the user's emotions and determines the priority of areas to be captured based on the estimated emotions. User emotions may include, for example, relaxation, tension, or being in a hurry, but are not limited to such examples. For example, if the user is relaxed, the camera unit prioritizes overall oral imaging. If the user is tense, the camera unit may prioritize imaging of specific areas. Furthermore, if the user is in a hurry, the camera unit may prioritize imaging of important areas. Thus, by determining the priority of areas to be captured according to the user's emotions, appropriate areas can be captured. Emotion estimation is realized using an emotion engine or generative AI, for example, with emotion estimation functions. Generative AI may estimate the user's emotions using technologies such as facial expression recognition and voice analysis. Some or all of the above-described processing in the camera unit may be performed using AI or without using AI. For example, the camera unit may input the user's facial expression data to generative AI and have the generative AI perform emotion estimation. Specifically, the camera unit acquires the user's facial images and voice data in real time and inputs them as multidimensional tensors (e.g., facial images: 3×224×224, voice spectrogram: 1×128×256) to an emotion estimation AI model (e.g., multimodal network combining CNN and RNN). Examples of AI input include “smiling face+calm voice” and “frowning face+high-pitched voice.” AI output includes emotion labels such as “relaxed,” “tense,” or “in a hurry,” and probability values for each emotion (e.g., relaxed 0.80, tense 0.15, in a hurry 0.05). Output examples include “relaxation level 0.85” and “tension level 0.65.” The camera unit uses these emotion estimation results to control the area priority decision module, such as “prioritize overall imaging when relaxed,” “prioritize imaging of abnormal areas when tense,” and “prioritize imaging of areas requiring treatment when in a hurry,” using rule-based control. Furthermore, the camera unit can accumulate the user's past emotional transitions and capture history in a time-series database and implement individually optimized area priority logic (e.g., automatically learning the optimal imaging order for each user). For AI model training, emotion-labeled facial images and voice datasets are used, and cross-entropy loss and data augmentation are utilized to improve accuracy. As a technical effect, the camera unit automates flexible selection of areas to be captured according to the user's psychological state, significantly improving imaging efficiency, user satisfaction, and diagnostic accuracy compared to conventional fixed imaging order or manual selection by the user. Application fields include self-care applications, dental diagnostic support, telemedicine, stress-free oral imaging for children and the elderly, and personalized healthcare services.

The camera unit can simultaneously collect audio data inside the mouth during image capture and detect abnormal sounds. For example, the camera unit simultaneously collects audio data inside the mouth during image capture and detects abnormal sounds. Collection of audio data may include, for example, the use of a microphone, but is not limited to such examples. The camera unit can detect the sound of teeth grinding during image capture. The camera unit can also detect abnormal chewing sounds during image capture. Furthermore, the camera unit can record and analyze abnormal sounds inside the mouth during image capture. Thus, by collecting audio data, abnormal sounds can be detected and the health condition inside the mouth can be evaluated. Some or all of the above-described processing in the camera unit may be performed using AI or without using AI. For example, the camera unit may input the collected audio data to generative AI and have the generative AI perform abnormal sound detection. Specifically, the camera unit uses a high-sensitivity microphone built into the toothbrush to acquire audio waveform data inside the mouth (e.g., 16 kHz sampling, 16,000 samples per second) simultaneously with image capture. Examples of input data include “waveform of teeth grinding sound,” “spectrogram of chewing sound,” and “normal conversation sound.” The camera unit preprocesses these audio data using feature extraction algorithms such as short-time Fourier transform (STFT) and Mel-frequency cepstral coefficients (MFCC), and inputs them to an abnormal sound detection AI model (e.g., 1D-CNN, RNN, self-supervised anomaly detection model). Examples of AI input include “MFCC features of teeth grinding sound” and “spectrogram of abnormal chewing sound.” AI output includes labels such as “normal,” “abnormal (teeth grinding),” “abnormal (chewing sound),” and abnormality scores (0.0 to 1.0). Output examples include “teeth grinding detection score 0.92” and “abnormal chewing sound score 0.78.” The camera unit records these outputs linked to image data and uses them for subsequent analysis, report generation for doctors, and lifestyle improvement proposals for users. For AI model training, audio datasets labeled with abnormal sounds are used, and cross-entropy loss and data augmentation (noise addition, pitch shift) are utilized to improve accuracy. As a technical effect, the camera unit achieves automatic detection, quantification, and recording of abnormal sounds, enabling multifaceted evaluation of oral health, early detection of abnormalities, and improved accuracy of treatment proposals compared to conventional diagnosis using only images or subjective evaluation by human hearing. Application fields include dental diagnostic support, self-care applications, monitoring of sleep apnea syndrome and teeth grinding, telemedicine, and medical big data analysis.

The camera unit can be equipped with an odor sensor inside the mouth during image capture and detect abnormal odors. For example, the camera unit is equipped with an odor sensor inside the mouth during image capture and detects abnormal odors. Equipping with an odor sensor may include, for example, the type of sensor and detection range, but is not limited to such examples. The camera unit can detect odors that cause bad breath during image capture. The camera unit can also detect and record abnormal odors during image capture. Furthermore, the camera unit can evaluate the health condition inside the mouth using an odor sensor. Thus, by equipping with an odor sensor, abnormal odors can be detected and the health condition inside the mouth can be evaluated. Some or all of the above-described processing in the camera unit may be performed using AI or without using AI. For example, the camera unit may input the detected odor data to generative AI and have the generative AI perform abnormal odor detection. Specifically, the camera unit uses semiconductor gas sensors or metal oxide sensors (e.g., VOC sensors, hydrogen sulfide sensors) built into the toothbrush to acquire concentrations of odor components inside the mouth (e.g., hydrogen sulfide, methyl mercaptan, ammonia) in real time during image capture. Examples of input data include “hydrogen sulfide concentration 2.5 ppm,” “VOC concentration 0.8 ppm,” and “time-series concentration data of multiple components.” The camera unit records these odor data linked to image data with timestamps and inputs them to an abnormal value detection AI model (e.g., multivariate anomaly detection neural network, decision tree, clustering algorithm). Examples of AI input include “hydrogen sulfide 2.5 ppm+gum image” and “VOC 0.8 ppm+tooth surface image.” AI output includes labels or scores such as “normal,” “abnormal (high risk of bad breath),” and “abnormality score 0.81.” Output examples include “bad breath risk score 0.85” and “normal range.” The camera unit integrates these outputs with image data and user history and uses them for subsequent analysis, report generation for doctors, and lifestyle improvement proposals for users. For AI model training, datasets labeled with odor components are used, and loss functions and data augmentation (noise addition to component concentrations) are utilized to improve accuracy. As a technical effect, the camera unit achieves quantitative and objective automatic detection and recording of odor components, enabling multifaceted evaluation of oral health, early detection of abnormalities, and improved accuracy of treatment proposals compared to conventional diagnosis relying on human olfaction or subjective evaluation. Application fields include dental diagnostic support, self-care applications, bad breath monitoring, telemedicine, and medical big data analysis.

The analysis unit can estimate the user's emotions and adjust the display method of analysis results based on the estimated emotions. For example, the analysis unit estimates the user's emotions and adjusts the display method of analysis results based on the estimated emotions. User emotions may include, for example, relaxation, tension, or being in a hurry, but are not limited to such examples. For example, if the user is relaxed, the analysis unit displays detailed analysis results. If the user is tense, the analysis unit may display concise analysis results. Furthermore, if the user is in a hurry, the analysis unit may display analysis results focusing on key points. Thus, by adjusting the display method of analysis results according to the user's emotions, appropriate information can be provided. Emotion estimation is realized using an emotion engine or generative AI, for example, with emotion estimation functions. Generative AI may estimate the user's emotions using technologies such as facial expression recognition and voice analysis. Some or all of the above-described processing in the analysis unit may be performed using AI or without using AI. For example, the analysis unit may input the user's facial expression data to generative AI and have the generative AI perform emotion estimation. Specifically, the analysis unit acquires the user's facial images (e.g., 3×224×224 RGB tensor) and voice spectrograms (e.g., 1×128×256 2D array) in real time and inputs them to a multimodal emotion estimation AI model (e.g., CNN+RNN or Transformer-based network). Examples of AI input include “smiling facial image+calm voice spectrogram” and “frowning face+tense high-pitched voice.” AI output includes emotion labels such as “relaxed,” “tense,” or “in a hurry” (e.g., outputting probability values for each emotion using softmax, e.g., relaxed 0.82, tense 0.12, in a hurry 0.06) and emotion intensity scores (continuous values from 0.0 to 1.0). Output examples include “relaxation level 0.85” and “tension level 0.65.” The analysis unit uses these emotion estimation results to control the analysis result display module, such as “if relaxation level is 0.7 or higher, display detailed analysis results,” “if tension level is 0.5 or higher, display only concise analysis results,” and “if being in a hurry level is 0.5 or higher, display only key points,” using rule-based control. Furthermore, the analysis unit can accumulate the user's past emotional transitions and analysis result viewing history in a time-series database and implement individually optimized display control logic (e.g., automatically learning the optimal amount and format of information for each user). For AI model training, emotion-labeled facial images and voice datasets are used, and cross-entropy loss and data augmentation (expression changes, voice noise addition) are utilized to improve accuracy. As a technical effect, the analysis unit automates flexible information presentation according to the user's psychological state, significantly improving user understanding, satisfaction, and behavioral change rate compared to conventional fixed analysis result display or manual selection by the user. Application fields include self-care applications, dental diagnostic support, telemedicine, stress-free presentation of diagnostic results for children and the elderly, and personalized healthcare services.

The analysis unit can optimize the analysis algorithm by referring to past analysis data during analysis. For example, the analysis unit optimizes the analysis algorithm by referring to past analysis data during analysis. Past analysis data may include, for example, database construction methods and types of data, but are not limited to such examples. The analysis unit can improve the accuracy of the algorithm based on past analysis data. The analysis unit can also enhance the reliability of analysis results by referring to past analysis data. Furthermore, the analysis unit can optimize the analysis algorithm using past analysis data. Thus, by referring to past analysis data, the accuracy of the analysis algorithm can be improved. Some or all of the above-described processing in the analysis unit may be performed using AI or without using AI. For example, the analysis unit may input past analysis data to generative AI and have the generative AI perform algorithm optimization. Specifically, the analysis unit acquires previously accumulated oral image datasets (e.g., tens of thousands to millions of RGB image tensors), analysis result labels (e.g., abnormal area labels, treatment history), and user attribute data (e.g., age, lifestyle vectors) from a distributed database and utilizes them for retraining and parameter optimization of AI models. Examples of AI input include “abnormality detection results for the past 1,000 cases+image tensors” and “past misclassification cases+corrected labels.” AI output includes “optimized weight parameter sets,” “new hyperparameter settings,” and “algorithm selection recommendations.” Output examples include “change the number of CNN layers from 4 to 6” and “optimize learning rate from 0.001 to 0.0005.” The analysis unit uses these outputs to automatically update the analysis algorithm, select models (e.g., switch from CNN to Vision Transformer), automatically correct misclassification patterns, and optimize data augmentation methods (e.g., adjust rotation and scaling rates for specific areas). Furthermore, the analysis unit can utilize online learning and transfer learning to continuously optimize the model as new data is added. For AI model training, loss functions (e.g., cross-entropy, Dice loss), weight optimization algorithms (e.g., Adam, SGD), and generalization performance evaluation using validation sets are combined. As a technical effect, the analysis unit achieves significant improvements in analysis accuracy, reproducibility, and reliability through automatic optimization using vast past data, compared to conventional static algorithms or manual parameter adjustment. Application fields include medical big data analysis, self-care applications, dental diagnostic support, personalized preventive medicine, and continuous AI model improvement services.

The analysis unit can introduce a scoring system for comprehensively evaluating the health condition inside the mouth during analysis. For example, the analysis unit introduces a scoring system for comprehensively evaluating the health condition inside the mouth during analysis. The scoring system may include, for example, methods for calculating scores and evaluation items, but is not limited to such examples. The analysis unit can score the health condition inside the mouth and provide a comprehensive evaluation. The analysis unit can also use the scoring system to evaluate the health condition inside the mouth in detail. Furthermore, the analysis unit can introduce a scoring system to display analysis results in an easy-to-understand manner. Thus, by introducing a scoring system, the health condition inside the mouth can be comprehensively evaluated. Some or all of the above-described processing in the analysis unit may be performed using AI or without using AI. For example, the analysis unit may input scoring system data to generative AI and have the generative AI perform evaluation. Specifically, the analysis unit quantifies multiple evaluation items such as the number of cavities, degree of progression of periodontal disease, amount of tartar deposition, degree of gum swelling, degree of tooth surface discoloration, oral temperature and humidity, abnormal sound and odor detection results, and inputs them as multidimensional vectors (e.g., 10-dimensional health indicator vector) to a scoring AI model (e.g., multilayer perceptron, decision tree, regression model). Examples of AI input include “number of cavities: 2, degree of progression of periodontal disease: 0.6, amount of tartar: 0.3, temperature: 36.5° C., humidity: 85%,” and “abnormal sound score: 0.8, odor risk: 0.7.” AI output includes “comprehensive health score (0-100 points),” “risk classification (low, medium, high),” and “health score for each area.” Output examples include “comprehensive score: 85 points,” “gum area score: 70 points,” and “risk classification: medium.” The analysis unit uses these scores to automatically generate comprehensive evaluation reports, area-specific improvement proposals, and comparison graphs with past scores, and displays them in an easy-to-understand manner on the user interface. Furthermore, the scoring system can implement personalized evaluation logic that takes into account the user's age, lifestyle, and past treatment history (e.g., age correction, lifestyle risk weighting). For AI model training, actual diagnostic results and treatment effect data are used as training data, and regression loss and classification loss are minimized. As a technical effect, the analysis unit automates objective and quantitative health evaluation integrating multiple indicators, strongly supporting user health management, preventive action, and treatment planning compared to conventional single-item evaluation or subjective diagnosis. Application fields include self-care applications, dental diagnostic support, telemedicine, personalized healthcare services, and health progress monitoring.

The analysis unit can estimate the user's emotions and determine the priority of analysis results based on the estimated emotions. For example, the analysis unit estimates the user's emotions and determines the priority of analysis results based on the estimated emotions. User emotions may include, for example, relaxation, tension, or being in a hurry, but are not limited to such examples. For example, if the user is relaxed, the analysis unit prioritizes displaying detailed analysis results. If the user is tense, the analysis unit may prioritize displaying concise analysis results. Furthermore, if the user is in a hurry, the analysis unit may prioritize displaying analysis results focusing on key points. Thus, by determining the priority of analysis results according to the user's emotions, appropriate information can be provided. Emotion estimation is realized using an emotion engine or generative AI, for example, with emotion estimation functions. Generative AI may estimate the user's emotions using technologies such as facial expression recognition and voice analysis. Some or all of the above-described processing in the analysis unit may be performed using AI or without using AI. For example, the analysis unit may input the user's facial expression data to generative AI and have the generative AI perform emotion estimation. Specifically, the analysis unit acquires the user's facial images (3×224×224 RGB tensor) and voice spectrograms (1×128×256) in real time and inputs them to a multimodal emotion estimation AI model (CNN +RNN or Transformer). Examples of AI input include “smiling face+calm voice” and “frowning face+high-pitched voice.” AI output includes emotion labels such as “relaxed,” “tense,” or “in a hurry,” and probability values for each emotion (e.g., relaxed 0.80, tense 0.15, in a hurry 0.05). Output examples include “relaxation level 0.85” and “tension level 0.65.” The analysis unit uses these emotion estimation results to control the analysis result priority decision module, such as “prioritize detailed analysis results when relaxed,” “prioritize concise analysis results when tense,” and “prioritize only key points when in a hurry,” using rule-based control. Furthermore, the analysis unit can accumulate the user's past emotional transitions and analysis result viewing history in a time-series database and implement individually optimized priority decision logic (e.g., automatically learning the optimal information presentation order for each user). For AI model training, emotion-labeled facial images and voice datasets are used, and cross-entropy loss and data augmentation are utilized to improve accuracy. As a technical effect, the analysis unit automates flexible information presentation order according to the user's psychological state, significantly improving user understanding, satisfaction, and behavioral change rate compared to conventional fixed information presentation order or manual selection by the user. Application fields include self-care applications, dental diagnostic support, telemedicine, stress-free presentation of diagnostic results, and personalized healthcare services.

The analysis unit can analyze the bacterial balance inside the mouth during analysis and detect abnormal balance. For example, the analysis unit analyzes the bacterial balance inside the mouth during analysis and detects abnormal balance. Analysis of bacterial balance may include, for example, types of bacteria and methods for evaluating balance, but is not limited to such examples. The analysis unit can analyze the bacterial balance inside the mouth and detect abnormal balance. The analysis unit can also propose appropriate treatment methods based on the results of bacterial balance analysis. Furthermore, the analysis unit can detect abnormal bacterial balance early and promote treatment. Thus, by analyzing bacterial balance, abnormal balance can be detected early and treatment can be promoted. Some or all of the above-described processing in the analysis unit may be performed using AI or without using AI. For example, the analysis unit may input bacterial balance data to generative AI and have the generative AI perform analysis. Specifically, the analysis unit integrates bacterial DNA sequence data obtained from the oral cavity (e.g., 16S rRNA sequence data, thousands to tens of thousands of base sequences), bacterial species ratio vectors (e.g., 30-dimensional bacterial species ratio vector), and image features (e.g., degree of gum swelling, amount of tartar) and inputs them to a multimodal AI model (e.g., neural network+clustering algorithm). Examples of AI input include “bacterial species ratio vector+gum image features” and “16S sequence data+amount of tartar.” AI output includes labels or scores such as “normal,” “abnormal (balance collapse),” and “risk score 0.78.” Output examples include “bacterial balance abnormality detection: risk 0.82” and “normal range.” The analysis unit uses these outputs to link abnormal balance detection results to the treatment proposal module and automatically propose treatment methods such as “recommend probiotics” and “recommend antimicrobial treatment.” Furthermore, the analysis unit analyzes time-series changes in bacterial balance to achieve early detection of abnormal trends and monitoring of treatment effects. For AI model training, datasets labeled with bacterial species and treatment effect data are used, and cross-entropy loss and clustering loss are utilized. As a technical effect, the analysis unit achieves automatic analysis of vast bacterial species data, early detection of abnormal balance, treatment proposals, and progress monitoring with high accuracy and efficiency compared to conventional culture methods or subjective evaluation by humans. Application fields include dental diagnostic support, self-care applications, telemedicine, oral bacterial monitoring, and medical big data analysis.

The analysis unit is capable of analyzing pigmentation inside the mouth during analysis and detecting abnormal pigmentation. For example, the analysis unit analyzes pigmentation inside the mouth during analysis and detects abnormal pigmentation. The analysis of pigmentation may include, for example, the types of pigments and methods for evaluating deposition, but is not limited thereto. The analysis unit, for example, analyzes pigmentation inside the mouth and detects abnormal pigmentation. Furthermore, the analysis unit may propose appropriate treatment methods based on the analysis results of pigmentation. Additionally, the analysis unit can detect abnormalities in pigmentation at an early stage and prompt treatment. Thus, by analyzing pigmentation, abnormal pigmentation can be detected early and treatment can be promoted. Some or all of the above-described processing in the analysis unit may be performed using AI or without using AI. For example, the analysis unit may input pigmentation data into a generative AI and have the generative AI perform the analysis. Specifically, the analysis unit inputs oral image tensors (e.g., 3×1080×1920), segmentation maps of pigmentation sites (two-dimensional arrays), and pigment component spectral data (e.g., hyperspectral image data) into an AI model (e.g., CNN plus spectral analysis network). Examples of AI input include “tooth surface image plus pigment spectrum” and “gingiva image plus deposition site mask.” The AI output may be obtained in formats such as “abnormal pigmentation detection label,” “deposition score (0.0-1.0),” and “treatment recommendation score.” Examples of output include “abnormal pigmentation score 0.81” and “treatment recommendation: whitening.” The analysis unit links these outputs to a treatment proposal module and automatically proposes treatment methods such as “cleaning recommendation” or “whitening recommendation” based on the abnormal deposition detection results. Furthermore, the analysis unit analyzes time-series changes in pigmentation, enabling early detection of abnormal trends and monitoring of treatment effects. For training the AI model, datasets of images labeled with pigmentation and treatment effect data are used, leveraging cross-entropy loss and regression loss. As a technical effect, the analysis unit can achieve highly accurate and efficient automatic detection, quantification, and treatment proposal for pigmentation compared to conventional visual diagnosis and subjective evaluation, greatly improving diagnostic accuracy, treatment effectiveness, and user satisfaction. Application fields include dental diagnostic support, self-care applications, telemedicine, oral pigmentation monitoring, and medical big data analysis.

The specifying unit is capable of estimating the user's emotions and adjusting the display method of identification results based on the estimated emotions. For example, the specifying unit estimates the user's emotions and adjusts the display method of identification results based on the estimated emotions. User emotions may include, for example, relaxation, tension, or being in a hurry, but are not limited thereto. For example, when the user is relaxed, the specifying unit displays detailed identification results. When the user is tense, the specifying unit may display concise identification results. Furthermore, when the user is in a hurry, the specifying unit may display identification results that focus on key points. Thus, by adjusting the display method of identification results according to the user's emotions, appropriate information can be provided. Emotion estimation may be realized using an emotion engine or generative AI, for example, by employing emotion estimation functions. Generative AI may estimate the user's emotions using technologies such as facial expression recognition and voice analysis. Some or all of the above-described processing in the specifying unit may be performed using AI or without using AI. For example, the specifying unit may input the user's facial expression data into a generative AI and have the generative AI perform emotion estimation. Specifically, the specifying unit can acquire the user's facial images (e.g., 3×224×224 RGB tensor) and voice spectrograms (e.g., 1×128×256 two-dimensional array) in real time and input them into a multimodal emotion estimation AI model (e.g., a combination of convolutional neural networks and recurrent neural networks, or a Transformer-based network). Examples of AI input include “smiling face image plus calm voice spectrogram” and “frowning face plus high-pitched tense voice.” The AI output may be obtained as emotion labels such as “relaxed,” “tense,” or “in a hurry” (e.g., outputting probability values for each emotion in a softmax probability distribution, e.g., relaxed 0.82, tense 0.12, in a hurry 0.06) or as emotion intensity scores (continuous values from 0.0 to 1.0). Examples of output include “relaxation score 0.85” and “tension score 0.65.” Based on these emotion estimation results, the identification result display control module in the specifying unit executes rule-based control such as “if relaxation score is 0.7 or higher, display detailed identification results,” “if tension score is 0.5 or higher, display only concise identification results,” and “if in a hurry score is 0.5 or higher, display only key points.” Furthermore, the specifying unit can accumulate the user's past emotional transitions and identification result viewing history in a time-series database and implement individually optimized display control logic (e.g., automatically learning the optimal amount and format of information for each user). For training the AI model, datasets of facial images and voice data labeled with emotions are used, and accuracy is improved by utilizing cross-entropy loss and data augmentation (such as expression changes and voice noise addition). Compared to conventional fixed identification result displays or manual user selection, the specifying unit can automate flexible information presentation according to the user's psychological state, greatly improving user comprehension, satisfaction, and behavior change rates. Application fields include self-care applications, dental diagnostic support, telemedicine, stress-free presentation of diagnostic results for children and the elderly, and personalized healthcare services.

The specifying unit is capable of optimizing the identification algorithm by referring to past identification data during identification. For example, the specifying unit optimizes the identification algorithm by referring to past identification data during identification. Past identification data may include, for example, database construction methods and types of data, but are not limited thereto. For example, the specifying unit improves the accuracy of the algorithm based on past identification data. Additionally, the specifying unit may enhance the reliability of identification results by referring to past identification data. Furthermore, the specifying unit may optimize the identification algorithm using past identification data. Thus, by referring to past identification data, the accuracy of the identification algorithm can be improved. Some or all of the above-described processing in the specifying unit may be performed using AI or without using AI. For example, the specifying unit may input past identification data into a generative AI and have the generative AI perform algorithm optimization. Specifically, the specifying unit acquires datasets of previously accumulated area(s) requiring treatment identification data (e.g., tens of thousands to millions of image tensors labeled with abnormal sites, identification result lists, treatment histories, user attribute vectors) from a distributed database and utilizes them for retraining the AI model or parameter optimization. Examples of AI input include “the last 1,000 identification results for areas requiring treatment plus image tensors” and “past misidentification cases plus correction labels.” The AI output may be obtained in formats such as “optimized weight parameter set,” “new hyperparameter settings,” and “algorithm selection recommendations.” Examples of output include “optimized branching conditions for decision trees,” “increase the number of trees in random forest from 100 to 200,” and “optimize learning rate from 0.001 to 0.0005.” Based on these outputs, the specifying unit performs automatic updates of the identification algorithm, model selection (e.g., switching from decision tree to random forest), automatic correction of misidentification patterns, and optimization of data augmentation methods (e.g., adjustment of rotation and scaling rates for specific areas). Furthermore, the specifying unit can utilize online learning and transfer learning to continuously optimize the model whenever new data is added. For training the AI model, loss functions (e.g., cross-entropy, F1 score optimization), weight optimization algorithms (e.g., Adam, SGD), and generalization performance evaluation using validation sets are combined. As a technical effect, the specifying unit can greatly improve identification accuracy, reproducibility, and reliability by automatic optimization utilizing vast past data, compared to conventional static algorithms or manual parameter adjustment. Application fields include medical big data analysis, self-care applications, dental diagnostic support, personalized preventive medicine, and continuous AI model improvement services.

The specifying unit is capable of introducing a scoring system for comprehensively evaluating the health condition inside the mouth during identification. For example, the specifying unit introduces a scoring system for comprehensively evaluating the health condition inside the mouth during identification. The scoring system may include, for example, methods for calculating scores and evaluation items, but is not limited thereto. For example, the specifying unit scores the health condition inside the mouth and provides a comprehensive evaluation. Additionally, the specifying unit may use the scoring system to evaluate the health condition inside the mouth in detail. Furthermore, the specifying unit may introduce a scoring system to display identification results in an easily understandable manner. Thus, by introducing a scoring system, the health condition inside the mouth can be comprehensively evaluated. Some or all of the above-described processing in the specifying unit may be performed using AI or without using AI. For example, the specifying unit may input scoring system data into a generative AI and have the generative AI perform the evaluation. Specifically, the specifying unit quantifies multiple evaluation items such as the number of cavities, degree of progression of periodontal disease, amount of tartar deposition, degree of gingival swelling, degree of tooth surface staining, oral temperature and humidity, and results of abnormal sound and odor detection, and inputs these as a multidimensional vector (e.g., a 10-dimensional health index vector) into a scoring AI model (e.g., multilayer perceptron, decision tree, regression model). Examples of AI input include “number of cavities: 2, periodontal disease progression: 0.6, tartar amount: 0.3, temperature: 36.5° C., humidity: 85%” and “abnormal sound score: 0.8, odor risk: 0.7.” The AI output may be obtained in formats such as “comprehensive health score (0-100 points),” “risk classification (low, medium, high),” and “site-specific health score.” Examples of output include “comprehensive score: 85 points,” “gingival site score: 70 points,” and “risk classification: medium.” Based on these scores, the specifying unit automatically generates comprehensive evaluation reports, site-specific improvement proposals, and comparison graphs with past scores, and displays them in an easily understandable manner on the user interface. Furthermore, the scoring system may implement personalized evaluation logic that takes into account the user's age, lifestyle habits, and past treatment history (e.g., age correction, lifestyle risk weighting). For training the AI model, actual diagnostic results and treatment effect data are used as training data, and regression loss and classification loss are minimized. As a technical effect, the specifying unit can automate objective and quantitative health evaluation integrating multiple indicators, compared to conventional single-item evaluation or subjective diagnosis, thereby strongly supporting user health management, preventive actions, and treatment planning. Application fields include self-care applications, dental diagnostic support, telemedicine, personalized healthcare services, and health progress monitoring.

The specifying unit is capable of estimating the user's emotions and determining the priority of identification results based on the estimated emotions. For example, the specifying unit estimates the user's emotions and determines the priority of identification results based on the estimated emotions. User emotions may include, for example, relaxation, tension, or being in a hurry, but are not limited thereto. For example, when the user is relaxed, the specifying unit prioritizes the display of detailed identification results. When the user is tense, the specifying unit may prioritize the display of concise identification results. Furthermore, when the user is in a hurry, the specifying unit may prioritize the display of identification results that focus on key points. Thus, by determining the priority of identification results according to the user's emotions, appropriate information can be provided. Emotion estimation may be realized using an emotion engine or generative AI, for example, by employing emotion estimation functions. Generative AI may estimate the user's emotions using technologies such as facial expression recognition and voice analysis. Some or all of the above-described processing in the specifying unit may be performed using AI or without using AI. For example, the specifying unit may input the user's facial expression data into a generative AI and have the generative AI perform emotion estimation. Specifically, the specifying unit acquires the user's facial images (3×224×224 RGB tensor) and voice spectrograms (1×128×256) in real time and inputs them into a multimodal emotion estimation AI model (CNN plus RNN or Transformer). Examples of AI input include “smiling face plus calm voice” and “frowning face plus high-pitched voice.” The AI output may be obtained as emotion labels such as “relaxed,” “tense,” or “in a hurry,” and probability values for each emotion (e.g., relaxed 0.80, tense 0.15, in a hurry 0.05). Examples of output include “relaxation score 0.85” and “tension score 0.65.” Based on these emotion estimation results, the identification result priority determination module in the specifying unit executes rule-based control such as “prioritize detailed identification results when relaxed,” “prioritize concise identification results when tense,” and “prioritize only key points when in a hurry.” Furthermore, the specifying unit can accumulate the user's past emotional transitions and identification result viewing history in a time-series database and implement individually optimized priority determination logic (e.g., automatically learning the optimal information presentation order for each user). For training the AI model, datasets of facial images and voice data labeled with emotions are used, and accuracy is improved by utilizing cross-entropy loss and data augmentation. As a technical effect, the specifying unit can automate flexible information presentation order according to the user's psychological state, compared to conventional fixed information presentation order or manual user selection, greatly improving user comprehension, satisfaction, and behavior change rates. Application fields include self-care applications, dental diagnostic support, telemedicine, stress-free presentation of diagnostic results, and personalized healthcare services.

The specifying unit is capable of identifying the bacterial balance inside the mouth during identification and detecting abnormal balance. For example, the specifying unit identifies the bacterial balance inside the mouth during identification and detects abnormal balance. The identification of bacterial balance may include, for example, types of bacteria and methods for evaluating balance, but is not limited thereto. For example, the specifying unit identifies the bacterial balance inside the mouth and detects abnormal balance. Furthermore, the specifying unit may propose appropriate treatment methods based on the identification results of bacterial balance. Additionally, the specifying unit can detect abnormalities in bacterial balance at an early stage and prompt treatment. Thus, by identifying bacterial balance, abnormal balance can be detected early and treatment can be promoted. Some or all of the above-described processing in the specifying unit may be performed using AI or without using AI. For example, the specifying unit may input bacterial balance data into a generative AI and have the generative AI perform identification. Specifically, the specifying unit integrates bacterial DNA sequence data obtained from the oral cavity (e.g., 16S rRNA sequence data, thousands to tens of thousands of base sequences), presence ratio vectors for each bacterial species (e.g., 30-dimensional bacterial species ratio vector), and image features (e.g., degree of gingival swelling, amount of tartar), and inputs them into a multimodal AI model (e.g., neural network plus clustering algorithm). Examples of AI input include “bacterial species ratio vector plus gingival image features” and “16S sequence data plus tartar amount.” The AI output may be obtained as labels or scores such as “normal,” “abnormal (balance collapse),” and “risk score 0.78.” Examples of output include “abnormal bacterial balance detected: risk 0.82” and “within normal range.” Based on these outputs, the specifying unit links abnormal balance detection results to a treatment proposal module and automatically proposes treatment methods such as “probiotics recommendation” or “antibacterial treatment recommendation.” Furthermore, the specifying unit analyzes time-series changes in bacterial balance, enabling early detection of abnormal trends and monitoring of treatment effects. For training the AI model, datasets labeled with bacterial species and treatment effect data are used, leveraging cross-entropy loss and clustering loss. As a technical effect, the specifying unit can automatically analyze vast bacterial species data and achieve highly accurate and efficient early detection of abnormal balance, treatment proposal, and progress monitoring, compared to conventional culture methods or subjective evaluation by humans. Application fields include dental diagnostic support, self-care applications, telemedicine, oral bacterial monitoring, and medical big data analysis.

The specifying unit is capable of identifying pigmentation inside the mouth during identification and detecting abnormal pigmentation. For example, the specifying unit identifies pigmentation inside the mouth during identification and detects abnormal pigmentation. The identification of pigmentation may include, for example, types of pigments and methods for evaluating deposition, but is not limited thereto. For example, the specifying unit identifies pigmentation inside the mouth and detects abnormal pigmentation. Furthermore, the specifying unit may propose appropriate treatment methods based on the identification results of pigmentation. Additionally, the specifying unit can detect abnormalities in pigmentation at an early stage and prompt treatment. Thus, by identifying pigmentation, abnormal pigmentation can be detected early and treatment can be promoted. Some or all of the above-described processing in the specifying unit may be performed using AI or without using AI. For example, the specifying unit may input pigmentation data into a generative AI and have the generative AI perform identification. Specifically, the specifying unit inputs oral image tensors (e.g., 3×1080×1920), segmentation maps of pigmentation sites (two-dimensional arrays), and pigment component spectral data (e.g., hyperspectral image data) into an AI model (e.g., CNN plus spectral analysis network). Examples of AI input include “tooth surface image plus pigment spectrum” and “gingiva image plus deposition site mask.” The AI output may be obtained in formats such as “abnormal pigmentation detection label,” “deposition score (0.0-1.0),” and “treatment recommendation score.” Examples of output include “abnormal pigmentation score 0.81” and “treatment recommendation: whitening.” The specifying unit links these outputs to a treatment proposal module and automatically proposes treatment methods such as “cleaning recommendation” or “whitening recommendation” based on the abnormal deposition detection results. Furthermore, the specifying unit analyzes time-series changes in pigmentation, enabling early detection of abnormal trends and monitoring of treatment effects. For training the AI model, datasets of images labeled with pigmentation and treatment effect data are used, leveraging cross-entropy loss and regression loss. As a technical effect, the specifying unit can achieve highly accurate and efficient automatic detection, quantification, and treatment proposal for pigmentation compared to conventional visual diagnosis and subjective evaluation, greatly improving diagnostic accuracy, treatment effectiveness, and user satisfaction. Application fields include dental diagnostic support, self-care applications, telemedicine, oral pigmentation monitoring, and medical big data analysis.

The generation unit is capable of estimating the user's emotions and adjusting the display method of generated images based on the estimated emotions. For example, the generation unit estimates the user's emotions and adjusts the display method of generated images based on the estimated emotions. User emotions may include, for example, relaxation, tension, or being in a hurry, but are not limited thereto. For example, when the user is relaxed, the generation unit displays detailed generated images. When the user is tense, the generation unit may display concise generated images. Furthermore, when the user is in a hurry, the generation unit may display generated images that focus on key points. Thus, by adjusting the display method of generated images according to the user's emotions, appropriate information can be provided. Emotion estimation may be realized using an emotion engine or generative AI, for example, by employing emotion estimation functions. Generative AI may estimate the user's emotions using technologies such as facial expression recognition and voice analysis. Some or all of the above-described processing in the generation unit may be performed using AI or without using AI. For example, the generation unit may input the user's facial expression data into a generative AI and have the generative AI perform emotion estimation. Specifically, the generation unit can acquire the user's facial images (3×224×224 RGB tensor) and voice spectrograms (1×128×256 two-dimensional array) in real time and input them into a multimodal emotion estimation AI model (a combination of convolutional neural networks and recurrent neural networks, or a Transformer-based network). Examples of AI input include “smiling face image plus calm voice spectrogram” and “frowning face plus high-pitched tense voice.” The AI output may be obtained as emotion labels such as “relaxed,” “tense,” or “in a hurry” (softmax probability distribution outputting probability values for each emotion, e.g., relaxed 0.82, tense 0.12, in a hurry 0.06) or as emotion intensity scores (continuous values from 0.0 to 1.0). Examples of output include “relaxation score 0.85” and “tension score 0.65.” Based on these emotion estimation results, the generated image display control module in the generation unit executes rule-based control such as “if relaxation score is 0.7 or higher, display detailed 3D models or treatment simulation images,” “if tension score is 0.5 or higher, display only concise 2D images highlighting abnormal areas,” and “if in a hurry score is 0.5 or higher, display only highlight images of areas requiring treatment.” Furthermore, the generation unit can accumulate the user's past emotional transitions and image viewing history in a time-series database and implement individually optimized display control logic (automatically learning the optimal amount and format of information for each user). For training the AI model, datasets of facial images and voice data labeled with emotions are used, and accuracy is improved by utilizing cross-entropy loss and data augmentation (such as expression changes and voice noise addition). Compared to conventional fixed image display or manual user selection, the generation unit can automate flexible information presentation according to the user's psychological state, greatly improving user comprehension, satisfaction, and treatment action rates. Application fields include self-care applications, dental diagnostic support, telemedicine, stress-free presentation of diagnostic results for children and the elderly, and personalized healthcare services.

The generation unit is capable of optimizing the generation algorithm by referring to past generation data during generation. For example, the generation unit optimizes the generation algorithm by referring to past generation data during generation. Past generation data may include, for example, database construction methods and types of data, but are not limited thereto. For example, the generation unit improves the accuracy of the algorithm based on past generation data. Additionally, the generation unit may enhance the reliability of generation results by referring to past generation data. Furthermore, the generation unit may optimize the generation algorithm using past generation data. Thus, by referring to past generation data, the accuracy of the generation algorithm can be improved. Some or all of the above-described processing in the generation unit may be performed using AI or without using AI. For example, the generation unit may input past generation data into a generative AI and have the generative AI perform algorithm optimization. Specifically, the generation unit acquires datasets of previously generated images of areas requiring treatment (e.g., tens of thousands to millions of 2D images, 3D mesh data, generation parameters, user evaluation labels) from a distributed database and utilizes them for retraining the AI model or parameter optimization. Examples of AI input include “the last 1,000 generated images plus user evaluation scores” and “past misgeneration cases plus correction parameters.” The AI output may be obtained in formats such as “optimized weight parameter set,” “new hyperparameter settings,” and “algorithm selection recommendations.” Examples of output include “optimized parameters for 3D reconstruction algorithm,” “increase the number of layers in the image generation model,” and “optimize learning rate from 0.001 to 0.0005.” Based on these outputs, the generation unit performs automatic updates of the generation algorithm, model selection (e.g., switching from 2D image generation to 3D model generation), automatic correction of misgeneration patterns, and optimization of data augmentation methods (adjustment of rotation and scaling rates for specific areas). Furthermore, the generation unit can utilize online learning and transfer learning to continuously optimize the model whenever new data is added. For training the AI model, loss functions (e.g., pixel error, IoU loss), weight optimization algorithms (Adam, SGD), and generalization performance evaluation using validation sets are combined. As a technical effect, the generation unit can greatly improve generation accuracy, reproducibility, and reliability by automatic optimization utilizing vast past data, compared to conventional static algorithms or manual parameter adjustment. Application fields include medical big data analysis, self-care applications, dental diagnostic support, personalized treatment planning, and continuous AI model improvement services.

The generation unit is capable of introducing a scoring system for comprehensively evaluating the health condition inside the mouth during generation. For example, the generation unit introduces a scoring system for comprehensively evaluating the health condition inside the mouth during generation. The scoring system may include, for example, methods for calculating scores and evaluation items, but is not limited thereto. For example, the generation unit scores the health condition inside the mouth and provides a comprehensive evaluation. Additionally, the generation unit may use the scoring system to evaluate the health condition inside the mouth in detail. Furthermore, the generation unit may introduce a scoring system to display generation results in an easily understandable manner. Thus, by introducing a scoring system, the health condition inside the mouth can be comprehensively evaluated. Some or all of the above-described processing in the generation unit may be performed using AI or without using AI. For example, the generation unit may input scoring system data into a generative AI and have the generative AI perform the evaluation. Specifically, the generation unit quantifies multiple evaluation items such as the number of cavities, degree of progression of periodontal disease, amount of tartar deposition, degree of gingival swelling, degree of tooth surface staining, oral temperature and humidity, and results of abnormal sound and odor detection, and inputs these as a multidimensional vector (e.g., a 10-dimensional health index vector) into a scoring AI model (e.g., multilayer perceptron, decision tree, regression model). Examples of AI input include “number of cavities: 2, periodontal disease progression: 0.6, tartar amount: 0.3, temperature: 36.5° C., humidity: 85%” and “abnormal sound score: 0.8, odor risk: 0.7.” The AI output may be obtained in formats such as “comprehensive health score (0-100 points),” “risk classification (low, medium, high),” and “site-specific health score.” Examples of output include “comprehensive score: 85 points,” “gingival site score: 70 points,” and “risk classification: medium.” Based on these scores, the generation unit automatically generates comprehensive evaluation reports, site-specific improvement proposals, and comparison graphs with past scores, and displays them in an easily understandable manner on the user interface. Furthermore, the scoring system may implement personalized evaluation logic that takes into account the user's age, lifestyle habits, and past treatment history (e.g., age correction, lifestyle risk weighting). For training the AI model, actual diagnostic results and treatment effect data are used as training data, and regression loss and classification loss are minimized. As a technical effect, the generation unit can automate objective and quantitative health evaluation integrating multiple indicators, compared to conventional single-item evaluation or subjective diagnosis, thereby strongly supporting user health management, preventive actions, and treatment planning. Application fields include self-care applications, dental diagnostic support, telemedicine, personalized healthcare services, and health progress monitoring.

The generation unit is capable of estimating the user's emotions and determining the priority of generated images based on the estimated emotions. For example, the generation unit estimates the user's emotions and determines the priority of generated images based on the estimated emotions. User emotions may include, for example, relaxation, tension, or being in a hurry, but are not limited thereto. For example, when the user is relaxed, the generation unit prioritizes the display of detailed generated images. When the user is tense, the generation unit may prioritize the display of concise generated images. Furthermore, when the user is in a hurry, the generation unit may prioritize the display of generated images that focus on key points. Thus, by determining the priority of generated images according to the user's emotions, appropriate information can be provided. Emotion estimation may be realized using an emotion engine or generative AI, for example, by employing emotion estimation functions. Generative AI may estimate the user's emotions using technologies such as facial expression recognition and voice analysis. Some or all of the above-described processing in the generation unit may be performed using AI or without using AI. For example, the generation unit may input the user's facial expression data into a generative AI and have the generative AI perform emotion estimation. Specifically, the generation unit acquires the user's facial images (3×224×224 RGB tensor) and voice spectrograms (1×128×256) in real time and inputs them into a multimodal emotion estimation AI model (CNN plus RNN or Transformer). Examples of AI input include “smiling face plus calm voice” and “frowning face plus high-pitched voice.” The AI output may be obtained as emotion labels such as “relaxed,” “tense,” or “in a hurry,” and probability values for each emotion (e.g., relaxed 0.80, tense 0.15, in a hurry 0.05). Examples of output include “relaxation score 0.85” and “tension score 0.65.” Based on these emotion estimation results, the generated image priority determination module in the generation unit executes rule-based control such as “prioritize detailed 3D models or treatment simulation images when relaxed,” “prioritize concise 2D images highlighting abnormal areas when tense,” and “prioritize only highlight images of areas requiring treatment when in a hurry.” Furthermore, the generation unit can accumulate the user's past emotional transitions and image viewing history in a time-series database and implement individually optimized priority determination logic (automatically learning the optimal information presentation order for each user). For training the AI model, datasets of facial images and voice data labeled with emotions are used, and accuracy is improved by utilizing cross-entropy loss and data augmentation. As a technical effect, the generation unit can automate flexible information presentation order according to the user's psychological state, compared to conventional fixed information presentation order or manual user selection, greatly improving user comprehension, satisfaction, and treatment action rates. Application fields include self-care applications, dental diagnostic support, telemedicine, stress-free presentation of diagnostic results, and personalized healthcare services.

The generation unit is capable of generating the bacterial balance inside the mouth during generation and detecting abnormal balance. For example, the generation unit generates the bacterial balance inside the mouth during generation and detects abnormal balance. The generation of bacterial balance may include, for example, types of bacteria and methods for evaluating balance, but is not limited thereto. For example, the generation unit generates the bacterial balance inside the mouth and detects abnormal balance. Furthermore, the generation unit may propose appropriate treatment methods based on the generation results of bacterial balance. Additionally, the generation unit can detect abnormalities in bacterial balance at an early stage and prompt treatment. Thus, by generating bacterial balance, abnormal balance can be detected early and treatment can be promoted. Some or all of the above-described processing in the generation unit may be performed using AI or without using AI. For example, the generation unit may input bacterial balance data into a generative AI and have the generative AI perform generation. Specifically, the generation unit integrates bacterial DNA sequence data obtained from the oral cavity (16S rRNA sequence data, thousands to tens of thousands of base sequences), presence ratio vectors for each bacterial species (30-dimensional bacterial species ratio vector), and image features (such as degree of gingival swelling and amount of tartar), and inputs them into a multimodal AI model (neural network plus clustering algorithm). Examples of AI input include “bacterial species ratio vector plus gingival image features” and “16S sequence data plus tartar amount.” The AI output may be obtained as labels or scores such as “normal,” “abnormal (balance collapse),” and “risk score 0.78.” Examples of output include “abnormal bacterial balance detected: risk 0.82” and “within normal range.” Based on these outputs, the generation unit links abnormal balance detection results to a treatment proposal module and automatically proposes treatment methods such as “probiotics recommendation” or “antibacterial treatment recommendation.” Furthermore, the generation unit generates time-series changes in bacterial balance, enabling early detection of abnormal trends and monitoring of treatment effects. For training the AI model, datasets labeled with bacterial species and treatment effect data are used, leveraging cross-entropy loss and clustering loss. As a technical effect, the generation unit can automatically generate and analyze vast bacterial species data and achieve highly accurate and efficient early detection of abnormal balance, treatment proposal, and progress monitoring, compared to conventional culture methods or subjective evaluation by humans. Application fields include dental diagnostic support, self-care applications, telemedicine, oral bacterial monitoring, and medical big data analysis.

The generation unit is capable of generating pigmentation inside the mouth during generation and detecting abnormal pigmentation. For example, the generation unit generates pigmentation inside the mouth during generation and detects abnormal pigmentation. The generation of pigmentation may include, for example, types of pigments and methods for evaluating deposition, but is not limited thereto. For example, the generation unit generates pigmentation inside the mouth and detects abnormal pigmentation. Furthermore, the generation unit may propose appropriate treatment methods based on the generation results of pigmentation. Additionally, the generation unit can detect abnormalities in pigmentation at an early stage and prompt treatment. Thus, by generating pigmentation, abnormal pigmentation can be detected early and treatment can be promoted. Some or all of the above-described processing in the generation unit may be performed using AI or without using AI. For example, the generation unit may input pigmentation data into a generative AI and have the generative AI perform generation. Specifically, the generation unit inputs oral image tensors (3×1080×1920), segmentation maps of pigmentation sites (two-dimensional arrays), and pigment component spectral data (hyperspectral image data) into an AI model (CNN plus spectral analysis network). Examples of AI input include “tooth surface image plus pigment spectrum” and “gingiva image plus deposition site mask.” The AI output may be obtained in formats such as “abnormal pigmentation detection label,” “deposition score (0.0-1.0),” and “treatment recommendation score.” Examples of output include “abnormal pigmentation score 0.81” and “treatment recommendation: whitening.” The generation unit links these outputs to a treatment proposal module and automatically proposes treatment methods such as “cleaning recommendation” or “whitening recommendation” based on the abnormal deposition detection results. Furthermore, the generation unit generates time-series changes in pigmentation, enabling early detection of abnormal trends and monitoring of treatment effects. For training the AI model, datasets of images labeled with pigmentation and treatment effect data are used, leveraging cross-entropy loss and regression loss. As a technical effect, the generation unit can achieve highly accurate and efficient automatic generation, quantification, and treatment proposal for pigmentation compared to conventional visual diagnosis and subjective evaluation, greatly improving diagnostic accuracy, treatment effectiveness, and user satisfaction. Application fields include dental diagnostic support, self-care applications, telemedicine, oral pigmentation monitoring, and medical big data analysis.

The sharing unit is capable of estimating the user's emotions and adjusting the display method of shared information based on the estimated emotions. For example, the sharing unit estimates the user's emotions and adjusts the display method of shared information based on the estimated emotions. User emotions may include, for example, relaxation, tension, or being in a hurry, but are not limited thereto. For example, when the user is relaxed, the sharing unit displays detailed shared information. When the user is tense, the sharing unit may display concise shared information. Furthermore, when the user is in a hurry, the sharing unit may display shared information that focuses on key points. Thus, by adjusting the display method of shared information according to the user's emotions, appropriate information can be provided. Emotion estimation may be realized using an emotion engine or generative AI, for example, by employing emotion estimation functions. Generative AI may estimate the user's emotions using technologies such as facial expression recognition and voice analysis. Some or all of the above-described processing in the sharing unit may be performed using AI or without using AI. For example, the sharing unit may input the user's facial expression data into a generative AI and have the generative AI perform emotion estimation. Specifically, the sharing unit can acquire the user's facial images (3×224×224 RGB tensor) and voice spectrograms (1×128×256 two-dimensional array) in real time and input them into a multimodal emotion estimation AI model (a combination of convolutional neural networks and recurrent neural networks, or a Transformer-based network). Examples of AI input include “smiling face image plus calm voice spectrogram” and “frowning face plus high-pitched tense voice.” The AI output may be obtained as emotion labels such as “relaxed,” “tense,” or “in a hurry” (softmax probability distribution outputting probability values for each emotion, e.g., relaxed 0.82, tense 0.12, in a hurry 0.06) or as emotion intensity scores (continuous values from 0.0 to 1.0). Examples of output include “relaxation score 0.85” and “tension score 0.65.” Based on these emotion estimation results, the shared information display control module in the sharing unit executes rule-based control such as “if relaxation score is 0.7 or higher, display detailed shared information,” “if tension score is 0.5 or higher, display only concise shared information,” and “if in a hurry score is 0.5 or higher, display only key points.” Furthermore, the sharing unit can accumulate the user's past emotional transitions and shared information viewing history in a time-series database and implement individually optimized display control logic (automatically learning the optimal amount and format of information for each user). For training the AI model, datasets of facial images and voice data labeled with emotions are used, and accuracy is improved by utilizing cross-entropy loss and data augmentation (such as expression changes and voice noise addition). Compared to conventional fixed information display or manual user selection, the sharing unit can automate flexible information presentation according to the user's psychological state, greatly improving user comprehension, satisfaction, and behavior change rates. Application fields include self-care applications, dental diagnostic support, telemedicine, stress-free presentation of diagnostic results for children and the elderly, and personalized healthcare services.

The sharing unit is capable of optimizing the sharing algorithm by referring to past sharing data during sharing. For example, the sharing unit optimizes the sharing algorithm by referring to past sharing data during sharing. Past sharing data may include, for example, database construction methods and types of data, but are not limited thereto. For example, the sharing unit improves the accuracy of the algorithm based on past sharing data. Additionally, the sharing unit may enhance the reliability of sharing results by referring to past sharing data. Furthermore, the sharing unit may optimize the sharing algorithm using past sharing data. Thus, by referring to past sharing data, the accuracy of the sharing algorithm can be improved. Some or all of the above-described processing in the sharing unit may be performed using AI or without using AI. For example, the sharing unit may input past sharing data into a generative AI and have the generative AI perform algorithm optimization. Specifically, the sharing unit acquires datasets of previously shared medical information (e.g., tens of thousands to millions of sharing histories, display formats, user evaluation scores, mis-sharing cases, correction histories) from a distributed database and utilizes them for retraining the AI model or parameter optimization. Examples of AI input include “the last 1,000 shared information items plus user evaluation scores” and “past mis-sharing cases plus correction parameters.” The AI output may be obtained in formats such as “optimized weight parameter set,” “new hyperparameter settings,” and “algorithm selection recommendations.” Examples of output include “optimized parameters for information display algorithm,” “increase the number of layers in the shared information classification model,” and “optimize learning rate from 0.001 to 0.0005.” Based on these outputs, the sharing unit performs automatic updates of the sharing algorithm, model selection (e.g., switching from rule-based to machine learning model), automatic correction of mis-sharing patterns, and optimization of data augmentation methods (adjustment of emphasis display rate for specific information). Furthermore, the sharing unit can utilize online learning and transfer learning to continuously optimize the model whenever new data is added. For training the AI model, loss functions (e.g., cross-entropy, F1 score optimization), weight optimization algorithms (Adam, SGD), and generalization performance evaluation using validation sets are combined. As a technical effect, the sharing unit can greatly improve sharing accuracy, reproducibility, and reliability by automatic optimization utilizing vast past data, compared to conventional static algorithms or manual parameter adjustment. Application fields include medical big data analysis, self-care applications, dental diagnostic support, personalized preventive medicine, and continuous AI model improvement services.

The sharing unit is capable of introducing a scoring system for comprehensively evaluating the health condition inside the mouth during sharing. For example, the sharing unit introduces a scoring system for comprehensively evaluating the health condition inside the mouth during sharing. The scoring system may include, for example, methods for calculating scores and evaluation items, but is not limited thereto. For example, the sharing unit scores the health condition inside the mouth and provides a comprehensive evaluation. Additionally, the sharing unit may use the scoring system to evaluate the health condition inside the mouth in detail. Furthermore, the sharing unit may introduce a scoring system to display sharing results in an easily understandable manner. Thus, by introducing a scoring system, the health condition inside the mouth can be comprehensively evaluated. Some or all of the above-described processing in the sharing unit may be performed using AI or without using AI. For example, the sharing unit may input scoring system data into a generative AI and have the generative AI perform the evaluation. Specifically, the sharing unit quantifies multiple evaluation items such as the number of cavities, degree of progression of periodontal disease, amount of tartar deposition, degree of gingival swelling, degree of tooth surface staining, oral temperature and humidity, and results of abnormal sound and odor detection, and inputs these as a multidimensional vector (e.g., a 10-dimensional health index vector) into a scoring AI model (e.g., multilayer perceptron, decision tree, regression model). Examples of AI input include “number of cavities: 2, periodontal disease progression: 0.6, tartar amount: 0.3, temperature: 36.5° C., humidity: 85%” and “abnormal sound score: 0.8, odor risk: 0.7.” The AI output may be obtained in formats such as “comprehensive health score (0-100 points),” “risk classification (low, medium, high),” and “site-specific health score.” Examples of output include “comprehensive score: 85 points,” “gingival site score: 70 points,” and “risk classification: medium.” Based on these scores, the sharing unit automatically generates comprehensive evaluation reports, site-specific improvement proposals, and comparison graphs with past scores, and displays them in an easily understandable manner on the user interface. Furthermore, the scoring system may implement personalized evaluation logic that takes into account the user's age, lifestyle habits, and past treatment history (e.g., age correction, lifestyle risk weighting). For training the AI model, actual diagnostic results and treatment effect data are used as training data, and regression loss and classification loss are minimized. As a technical effect, the sharing unit can automate objective and quantitative health evaluation integrating multiple indicators, compared to conventional single-item evaluation or subjective diagnosis, thereby strongly supporting user health management, preventive actions, and treatment planning. Application fields include self-care applications, dental diagnostic support, telemedicine, personalized healthcare services, and health progress monitoring.

The sharing unit is capable of estimating the user's emotions and determining the priority of shared information based on the estimated emotions. For example, the sharing unit estimates the user's emotions and determines the priority of shared information based on the estimated emotions. User emotions may include, for example, relaxation, tension, or being in a hurry, but are not limited thereto. For example, when the user is relaxed, the sharing unit prioritizes the display of detailed shared information. When the user is tense, the sharing unit may prioritize the display of concise shared information. Furthermore, when the user is in a hurry, the sharing unit may prioritize the display of shared information that focuses on key points. Thus, by determining the priority of shared information according to the user's emotions, appropriate information can be provided. Emotion estimation may be realized using an emotion engine or generative AI, for example, by employing emotion estimation functions. Generative AI may estimate the user's emotions using technologies such as facial expression recognition and voice analysis. Some or all of the above-described processing in the sharing unit may be performed using AI or without using AI. For example, the sharing unit may input the user's facial expression data into a generative AI and have the generative AI perform emotion estimation. Specifically, the sharing unit acquires the user's facial images (3×224×224 RGB tensor) and voice spectrograms (1×128×256) in real time and inputs them into a multimodal emotion estimation AI model (CNN plus RNN or Transformer). Examples of AI input include “smiling face plus calm voice” and “frowning face plus high-pitched voice.” The AI output may be obtained as emotion labels such as “relaxed,” “tense,” or “in a hurry,” and probability values for each emotion (e.g., relaxed 0.80, tense 0.15, in a hurry 0.05). Examples of output include “relaxation score 0.85” and “tension score 0.65.” Based on these emotion estimation results, the shared information priority determination module in the sharing unit executes rule-based control such as “prioritize detailed information when relaxed,” “prioritize concise information when tense,” and “prioritize only key points when in a hurry.” Furthermore, the sharing unit can accumulate the user's past emotional transitions and shared information viewing history in a time-series database and implement individually optimized priority determination logic (automatically learning the optimal information presentation order for each user). For training the AI model, datasets of facial images and voice data labeled with emotions are used, and accuracy is improved by utilizing cross-entropy loss and data augmentation. As a technical effect, the sharing unit can automate flexible information presentation order according to the user's psychological state, compared to conventional fixed information presentation order or manual user selection, greatly improving user comprehension, satisfaction, and behavior change rates. Application fields include self-care applications, dental diagnostic support, telemedicine, stress-free presentation of diagnostic results, and personalized healthcare services.

The sharing unit is capable of sharing the bacterial balance inside the mouth during sharing and detecting abnormal balance. For example, the sharing unit shares the bacterial balance inside the mouth during sharing and detects abnormal balance. The sharing of bacterial balance may include, for example, types of bacteria and methods for evaluating balance, but is not limited thereto. For example, the sharing unit shares the bacterial balance inside the mouth and detects abnormal balance. Furthermore, the sharing unit may propose appropriate treatment methods based on the sharing results of bacterial balance. Additionally, the sharing unit can detect abnormalities in bacterial balance at an early stage and prompt treatment. Thus, by sharing bacterial balance, abnormal balance can be detected early and treatment can be promoted. Some or all of the above-described processing in the sharing unit may be performed using AI or without using AI. For example, the sharing unit may input bacterial balance data into a generative AI and have the generative AI perform sharing. Specifically, the sharing unit integrates bacterial DNA sequence data obtained from the oral cavity (16S rRNA sequence data, thousands to tens of thousands of base sequences), presence ratio vectors for each bacterial species (30-dimensional bacterial species ratio vector), and image features (such as degree of gingival swelling and amount of tartar), and inputs them into a multimodal AI model (neural network plus clustering algorithm). Examples of AI input include “bacterial species ratio vector plus gingival image features” and “16S sequence data plus tartar amount.” The AI output may be obtained as labels or scores such as “normal,” “abnormal (balance collapse),” and “risk score 0.78.” Examples of output include “abnormal bacterial balance detected: risk 0.82” and “within normal range.” Based on these outputs, the sharing unit links abnormal balance detection results to a treatment proposal module and automatically proposes treatment methods such as “probiotics recommendation” or “antibacterial treatment recommendation.” Furthermore, the sharing unit shares time-series changes in bacterial balance, enabling early detection of abnormal trends and monitoring of treatment effects. For training the AI model, datasets labeled with bacterial species and treatment effect data are used, leveraging cross-entropy loss and clustering loss. As a technical effect, the sharing unit can automatically share and analyze vast bacterial species data and achieve highly accurate and efficient early detection of abnormal balance, treatment proposal, and progress monitoring, compared to conventional culture methods or subjective evaluation by humans. Application fields include dental diagnostic support, self-care applications, telemedicine, oral bacterial monitoring, and medical big data analysis.

The sharing unit is capable of sharing pigmentation inside the mouth during sharing and detecting abnormal pigmentation. For example, the sharing unit shares pigmentation inside the mouth during sharing and detects abnormal pigmentation. The sharing of pigmentation may include, for example, types of pigments and methods for evaluating deposition, but is not limited thereto. For example, the sharing unit shares pigmentation inside the mouth and detects abnormal pigmentation. Furthermore, the sharing unit may propose appropriate treatment methods based on the sharing results of pigmentation. Additionally, the sharing unit can detect abnormalities in pigmentation at an early stage and prompt treatment. Thus, by sharing pigmentation, abnormal pigmentation can be detected early and treatment can be promoted. Some or all of the above-described processing in the sharing unit may be performed using AI or without using AI. For example, the sharing unit may input pigmentation data into a generative AI and have the generative AI perform sharing. Specifically, the sharing unit inputs oral image tensors (3×1080×1920), segmentation maps of pigmentation sites (two-dimensional arrays), and pigment component spectral data (hyperspectral image data) into an AI model (CNN plus spectral analysis network). Examples of AI input include “tooth surface image plus pigment spectrum” and “gingiva image plus deposition site mask.” The AI output may be obtained in formats such as “abnormal pigmentation detection label,” “deposition score (0.0-1.0),” and “treatment recommendation score.” Examples of output include “abnormal pigmentation score 0.81” and “treatment recommendation: whitening.” The sharing unit links these outputs to a treatment proposal module and automatically proposes treatment methods such as “cleaning recommendation” or “whitening recommendation” based on the abnormal deposition detection results. Furthermore, the sharing unit shares time-series changes in pigmentation, enabling early detection of abnormal trends and monitoring of treatment effects. For training the AI model, datasets of images labeled with pigmentation and treatment effect data are used, leveraging cross-entropy loss and regression loss. As a technical effect, the sharing unit can achieve highly accurate and efficient automatic sharing, quantification, and treatment proposal for pigmentation compared to conventional visual diagnosis and subjective evaluation, greatly improving diagnostic accuracy, treatment effectiveness, and user satisfaction. Application fields include dental diagnostic support, self-care applications, telemedicine, oral pigmentation monitoring, and medical big data analysis.

The data analysis unit is capable of estimating the user's emotions and adjusting the display method of analysis results based on the estimated emotions. For example, the data analysis unit estimates the user's emotions and adjusts the display method of analysis results based on the estimated emotions. User emotions may include, for example, relaxation, tension, or being in a hurry, but are not limited thereto. For example, when the user is relaxed, the data analysis unit displays detailed analysis results. When the user is tense, the data analysis unit may display concise analysis results. Furthermore, when the user is in a hurry, the data analysis unit may display analysis results that focus on key points. Thus, by adjusting the display method of analysis results according to the user's emotions, appropriate information can be provided. Emotion estimation may be realized using an emotion engine or generative AI, for example, by employing emotion estimation functions. Generative AI may estimate the user's emotions using technologies such as facial expression recognition and voice analysis. Some or all of the above-described processing in the data analysis unit may be performed using AI or without using AI. For example, the data analysis unit may input the user's facial expression data into a generative AI and have the generative AI perform emotion estimation. Specifically, the data analysis unit can acquire the user's facial images (e.g., 3×224×224 RGB tensor) and voice spectrograms (e.g., 1×128×256 two-dimensional array) in real time and input them into a multimodal emotion estimation AI model (e.g., a combination of convolutional neural networks and recurrent neural networks, or a Transformer-based network). Examples of AI input include “smiling face image plus calm voice spectrogram” and “frowning face plus high-pitched tense voice.” For these input data, the data analysis unit extracts facial expression features from facial images (e.g., shape vectors of eyes and mouth corners, facial landmark coordinates) and acoustic features from voice spectrograms (e.g., pitch, formant, energy distribution), and inputs them as integrated feature vectors into the AI model. The AI model, for example, extracts image features with CNN, analyzes time-series voice features with RNN or Transformer, and finally integrates all features for emotion classification in a fully connected layer. The AI output may be obtained as emotion labels such as “relaxed,” “tense,” or “in a hurry” (softmax probability distribution outputting probability values for each emotion, e.g., relaxed 0.82, tense 0.12, in a hurry 0.06) or as emotion intensity scores (continuous values from 0.0 to 1.0). Examples of output include “relaxation score 0.85” and “tension score 0.65.” Based on these emotion estimation results, the analysis result display control module in the data analysis unit executes rule-based control such as “if relaxation score is 0.7 or higher, display detailed analysis results,” “if tension score is 0.5 or higher, display only concise analysis results,” and “if in a hurry score is 0.5 or higher, display only key points.” Furthermore, the data analysis unit can accumulate the user's past emotional transitions and analysis result viewing history in a time-series database and implement individually optimized display control logic (automatically learning the optimal amount and format of information for each user). For training the AI model, datasets of facial images and voice data labeled with emotions are used, and accuracy is improved by utilizing cross-entropy loss and data augmentation (such as expression changes and voice noise addition). Compared to conventional fixed analysis result display or manual user selection, the data analysis unit can automate flexible information presentation according to the user's psychological state, greatly improving user comprehension, satisfaction, and behavior change rates. Application fields include self-care applications, dental diagnostic support, telemedicine, stress-free presentation of diagnostic results for children and the elderly, and personalized healthcare services.

The analysis unit can optimize the analysis algorithm by referring to past analysis data during analysis. For example, the analysis unit optimizes the analysis algorithm by referring to past analysis data during analysis. Past analysis data may include, for example, database construction methods and types of data, but is not limited to such examples. The analysis unit may, for example, improve the accuracy of the algorithm based on past analysis data. In addition, the analysis unit can enhance the reliability of analysis results by referring to past analysis data. Furthermore, the analysis unit can optimize the analysis algorithm using past analysis data. By referring to past analysis data, the accuracy of the analysis algorithm can be improved. Some or all of the above-described processing in the analysis unit may be performed using AI or without using AI. For example, the analysis unit may input past analysis data into generative AI and have the generative AI perform algorithm optimization. Specifically, the analysis unit acquires accumulated oral image datasets (e.g., tens of thousands to millions of RGB image tensors), analysis result labels (e.g., abnormal site labels, treatment history), and user attribute data (e.g., age, lifestyle habit vectors) from a distributed database, and utilizes these for retraining AI models and parameter optimization. Examples of input to AI include “abnormal detection results of the past 1,000 cases+image tensors” and “past misclassification cases+corrected labels.” The analysis unit uses these input data to perform retraining (fine-tuning) of AI model weight parameters, automatic search for hyperparameters (e.g., optimization of learning rate, batch size, number of network layers), and algorithm selection (e.g., switching from CNN to Vision Transformer). The output from AI is obtained in formats such as “optimized weight parameter set,” “new hyperparameter settings,” and “algorithm selection recommendations.” Examples of output include “change the number of CNN layers from 4 to 6” and “optimize the learning rate from 0.001 to 0.0005.” Based on these outputs, the analysis unit automatically updates the analysis algorithm, automatically corrects misclassification patterns, and optimizes data augmentation methods (e.g., adjustment of rotation and magnification rates for specific areas). Furthermore, the analysis unit can utilize online learning and transfer learning to continuously optimize the model as new data is added. For AI model training, combinations such as loss functions (e.g., cross-entropy, Dice loss), weight optimization algorithms (Adam, SGD), and generalization performance evaluation using validation sets are used. As a technical effect, the analysis unit can significantly improve analysis accuracy, reproducibility, and reliability through automatic optimization utilizing vast past data, compared to conventional static algorithms and manual parameter adjustment. Application fields include medical big data analysis, self-care applications, dental diagnostic support, personalized preventive medicine, and continuous AI model improvement services.

The analysis unit can introduce a scoring system for comprehensively evaluating the health condition inside the mouth during analysis. For example, the analysis unit introduces a scoring system for comprehensively evaluating the health condition inside the mouth during analysis. The scoring system may include, for example, methods for calculating scores and evaluation items, but is not limited to such examples. The analysis unit may score the health condition inside the mouth and provide a comprehensive evaluation. In addition, the analysis unit can use the scoring system to evaluate the health condition inside the mouth in detail. Furthermore, the analysis unit can introduce a scoring system to display analysis results in an easy-to-understand manner. By introducing a scoring system, the health condition inside the mouth can be comprehensively evaluated. Some or all of the above-described processing in the analysis unit may be performed using AI or without using AI. For example, the analysis unit may input scoring system data into generative AI and have the generative AI perform the evaluation. Specifically, the analysis unit quantifies multiple evaluation items such as the number of cavities, degree of progression of periodontal disease, amount of tartar deposition, degree of gum swelling, degree of tooth surface pigmentation, oral temperature and humidity, and abnormal sound/odor detection results, and inputs these as multidimensional vectors (e.g., 10-dimensional health indicator vectors) into a scoring AI model (e.g., multilayer perceptron, decision tree, regression model). Examples of input to AI include “number of cavities: 2, periodontal disease progression: 0.6, tartar amount: 0.3, temperature: 36.5° C., humidity: 85%” and “abnormal sound score: 0.8, odor risk: 0.7.” The analysis unit normalizes and standardizes these input data and inputs them into the AI model to allow learning of the weighting and correlations of each indicator. The output from AI is obtained in formats such as “comprehensive health score (0-100 points),” “risk classification (low, medium, high),” and “site-specific health score.” Examples of output include “comprehensive score: 85 points,” “gum site score: 70 points,” and “risk classification: medium.” Based on these scores, the analysis unit automatically generates comprehensive evaluation reports, site-specific improvement suggestions, and comparison graphs with past scores, and displays them in an easy-to-understand manner on the user interface. Furthermore, the scoring system can implement personalized evaluation logic (e.g., age correction, lifestyle risk weighting) that takes into account the user's age, lifestyle habits, and past treatment history. For AI model training, actual diagnostic results and treatment effect data are used as training data, and regression loss and classification loss are minimized. As a technical effect, the analysis unit can automate objective and quantitative health evaluation integrating multiple indicators, compared to conventional single-item evaluation and subjective diagnosis, and strongly support user health management, preventive actions, and treatment planning. Application fields include self-care applications, dental diagnostic support, telemedicine, personalized healthcare services, and health progress monitoring.

The analysis unit can estimate the user's emotions and determine the priority of analysis results based on the estimated emotions. For example, the analysis unit estimates the user's emotions and determines the priority of analysis results based on the estimated emotions. User emotions may include, for example, relaxation, tension, and being in a hurry, but are not limited to such examples. For example, when the user is relaxed, the analysis unit prioritizes the display of detailed analysis results. In addition, when the user is tense, the analysis unit can prioritize the display of concise analysis results. Furthermore, when the user is in a hurry, the analysis unit can prioritize the display of analysis results that focus on key points. By determining the priority of analysis results according to the user's emotions, appropriate information can be provided. Emotion estimation is realized, for example, by using an emotion engine or generative AI with emotion estimation functions. Generative AI estimates the user's emotions using technologies such as facial expression recognition and voice analysis. Some or all of the above-described processing in the analysis unit may be performed using AI or without using AI. For example, the analysis unit may input the user's facial expression data into generative AI and have the generative AI perform emotion estimation. Specifically, the analysis unit acquires the user's facial images (3×224×224 RGB tensors) and voice spectrograms (1×128×256) in real time, and inputs these into a multimodal emotion estimation AI model (CNN+RNN or Transformer). Examples of input to AI include “smiling face+calm voice” and “frowning face+high-pitched voice.” The analysis unit extracts facial features and acoustic features from these input data and performs emotion classification with the AI model. The output from AI is obtained as emotion labels such as “relaxed,” “tense,” and “in a hurry,” and probability values for each emotion (e.g., relaxed: 0.80, tense: 0.15, in a hurry: 0.05). Examples of output include “relaxation level: 0.85” and “tension level: 0.65.” Based on these emotion estimation results, the analysis result priority determination module executes rule-based control such as “prioritize detailed analysis results when relaxed,” “prioritize concise analysis results when tense,” and “prioritize only key points when in a hurry.” Furthermore, the user's past emotional transitions and analysis result viewing history are accumulated in a time-series database, and individually optimized priority determination logic (automatic learning of optimal information presentation order for each user) can also be implemented. For AI model training, facial images and voice datasets labeled with emotions are used, and cross-entropy loss and data augmentation are utilized to improve accuracy. As a technical effect, the analysis unit can automate flexible information presentation order according to the user's psychological state, compared to conventional fixed information presentation order and manual user selection, and greatly improve user understanding, satisfaction, and behavioral change rate. Application fields include self-care applications, dental diagnostic support, telemedicine, stress-free presentation of diagnostic results, and personalized healthcare services.

The analysis unit can analyze the bacterial balance inside the mouth during analysis and detect abnormal balance. For example, the analysis unit analyzes the bacterial balance inside the mouth during analysis and detects abnormal balance. Bacterial balance analysis may include, for example, types of bacteria and methods for evaluating balance, but is not limited to such examples. For example, the analysis unit analyzes the bacterial balance inside the mouth and detects abnormal balance. In addition, the analysis unit can propose appropriate treatment methods based on the results of bacterial balance analysis. Furthermore, the analysis unit can detect abnormal bacterial balance at an early stage and prompt treatment. By analyzing bacterial balance, abnormal balance can be detected early and treatment can be prompted. Some or all of the above-described processing in the analysis unit may be performed using AI or without using AI. For example, the analysis unit may input bacterial balance data into generative AI and have the generative AI perform the analysis. Specifically, the analysis unit integrates bacterial DNA sequence data obtained from the oral cavity (e.g., 16S rRNA sequence data, thousands to tens of thousands of base sequences), presence ratio vectors for each bacterial species (e.g., 30-dimensional bacterial species ratio vector), and image features (e.g., degree of gum swelling, amount of tartar), and inputs them into a multimodal AI model (e.g., neural network+clustering algorithm). Examples of input to AI include “bacterial species ratio vector+gum image features” and “16S sequence data+tartar amount.” The analysis unit uses these input data to extract diversity indices of bacterial species (e.g., Shannon entropy) and increase/decrease patterns of specific bacterial species, and performs normal/abnormal judgment and risk score calculation with the AI model. The output from AI is obtained as labels and scores such as “normal,” “abnormal (balance collapse),” and “risk score: 0.78.” Examples of output include “bacterial balance abnormality detected: risk 0.82” and “within normal range.” Based on these outputs, the analysis unit links abnormal balance detection results to the treatment proposal module and automatically proposes treatment methods such as “probiotics recommended” and “antibacterial treatment recommended.” Furthermore, the analysis unit analyzes time-series changes in bacterial balance to realize early detection of abnormal trends and monitoring of treatment effects. For AI model training, datasets labeled with bacterial species and treatment effect data are used, and cross-entropy loss and clustering loss are utilized. As a technical effect, the analysis unit can automatically analyze vast bacterial species data and realize early detection of abnormal balance, treatment proposals, and progress monitoring with high accuracy and efficiency, compared to conventional culture methods and subjective evaluation by humans. Application fields include dental diagnostic support, self-care applications, telemedicine, oral bacterial monitoring, and medical big data analysis.

The analysis unit can analyze pigmentation inside the mouth during analysis and detect abnormal pigmentation. For example, the analysis unit analyzes pigmentation inside the mouth during analysis and detects abnormal pigmentation. Pigmentation analysis may include, for example, types of pigments and methods for evaluating deposition, but is not limited to such examples. For example, the analysis unit analyzes pigmentation inside the mouth and detects abnormal pigmentation. In addition, the analysis unit can propose appropriate treatment methods based on the results of pigmentation analysis. Furthermore, the analysis unit can detect abnormal pigmentation at an early stage and prompt treatment. By analyzing pigmentation, abnormal pigmentation can be detected early and treatment can be prompted. Some or all of the above-described processing in the analysis unit may be performed using AI or without using AI. For example, the analysis unit may input pigmentation data into generative AI and have the generative AI perform the analysis. Specifically, the analysis unit inputs oral image tensors (e.g., 3×1080×1920), segmentation maps of pigmentation sites (2D arrays), and pigment component spectral data (e.g., spectral image data) into an AI model (e.g., CNN+spectral analysis network). Examples of input to AI include “tooth surface image+pigment spectrum” and “gum image+deposition site mask.” The analysis unit extracts pigment distribution and spectral features for each image region from these input data and calculates the presence or absence of abnormal deposition and deposition score with the AI model. The output from AI is obtained in formats such as “abnormal pigmentation detection label,” “deposition score (0.0-1.0),” and “treatment recommendation level.” Examples of output include “abnormal pigmentation score: 0.81” and “treatment recommended: whitening.” Based on these outputs, the analysis unit links abnormal deposition detection results to the treatment proposal module and automatically proposes treatment methods such as “cleaning recommended” and “whitening recommended.” Furthermore, the analysis unit analyzes time-series changes in pigmentation to realize early detection of abnormal trends and monitoring of treatment effects. For AI model training, image datasets labeled with pigmentation and treatment effect data are used, and cross-entropy loss and regression loss are utilized. As a technical effect, the analysis unit can realize automatic detection, quantification, and treatment proposals for pigmentation with high accuracy and efficiency, compared to conventional visual diagnosis and subjective evaluation, and greatly improve diagnostic accuracy, treatment effect, and user satisfaction. Application fields include dental diagnostic support, self-care applications, telemedicine, oral pigmentation monitoring, and medical big data analysis.

The system according to the embodiment is not limited to the above-described examples and can be variously modified as follows, for example. Specifically, the system can flexibly change the module configuration and data flow of the camera unit, analysis unit, specifying unit, generation unit, sharing unit, and analysis unit. For example, the camera unit can implement a movable camera arm, simultaneous control of multiple cameras, addition of infrared/ultraviolet imaging functions, and linkage with super-resolution image generation AI. The analysis unit can adopt a multimodal AI architecture that integrates various sensor data such as image analysis AI, voice, odor, temperature/humidity, and biological signals. The specifying unit can improve the accuracy of identifying areas requiring treatment by considering user lifestyle habits, genetic information, medication history, and by ensemble inference of multiple models for risk prediction AI. The generation unit can add support for 3D mesh generation, treatment simulation video generation, and augmented reality (AR) display in addition to 2D image generation. The sharing unit can implement secure data linkage between medical institutions, information sharing with authority management among patients, families, and doctors, and real-time remote diagnostic support. The analysis unit can introduce advanced analysis algorithms such as big data analysis infrastructure, cloud distributed learning, anomaly detection AI, time-series prediction AI, and causal inference AI. Furthermore, each unit's AI model can support various learning methods such as distributed inference on GPU clusters or edge devices, online learning, transfer learning, and self-supervised learning. With these configuration changes, the system can greatly improve flexibility, scalability, accuracy, real-time performance, and user adaptability compared to conventional single-function diagnostic devices, and can be applied to various fields such as dental diagnostic support, self-care, telemedicine, medical big data analysis, and personalized healthcare services.

The camera unit can track the movement inside the user's mouth in real time when capturing images and automatically adjust the position and angle of the camera according to the movement. For example, when the user moves the toothbrush with their mouth open, the camera unit maintains the optimal shooting position in accordance with the movement. In addition, when the user closes their mouth, the camera unit can temporarily pause shooting and resume shooting when the mouth is opened again. Furthermore, the camera unit can predict the movement inside the user's mouth and pre-adjust the camera position in preparation for the next movement. As a result, optimal shooting according to the movement inside the user's mouth is possible, and detailed images can be obtained. Specifically, the camera unit acquires multiple sensor data such as accelerometer, gyroscope, toothbrush motion sensor, and intraoral electromyography sensor at a sampling rate of 100 Hz or higher per second to detect intraoral movement in real time. These sensor data (e.g., 3-axis acceleration vector, angular velocity vector, scalar value of mouth opening/closing degree) are input as time-series arrays into a camera control AI model (e.g., LSTM-based time-series prediction network, reinforcement learning agent). Examples of input to AI include “time-series data of acceleration and angular velocity for the past 2 seconds” and “continuous value array of mouth opening/closing degree.” The camera unit automatically recognizes intraoral motion patterns (e.g., opening, closing, chewing, brushing motion) from these input data and outputs control signals for next motion prediction and optimal camera position/angle. The output from AI is obtained in formats such as “camera position coordinates (x, y, z),” “camera angle (pitch, yaw, roll),” and “shooting start/stop flag.” Examples of output include “camera position (12, 8, 5), angle (10°, 0°, 5°), shooting ON” and “camera position (0, 0, 0), shooting OFF.” Based on these outputs, the servo motor control module adjusts the physical position and angle of the camera in real time to achieve optimal shooting that follows the user's movement. Furthermore, the AI model can accumulate user-specific motion history and intraoral shape data and implement individually optimized camera control logic (e.g., automatic learning of optimal tracking parameters for each user). As a technical effect, the camera unit can automate high-precision shooting that responds immediately to user movement, compared to conventional fixed cameras and manual adjustment, and greatly improve diagnostic accuracy, user convenience, and image data quality. Application fields include dental diagnostic support, self-care applications, telemedicine, intraoral motion analysis, and medical robotics.

The analysis unit can calculate a health score for comprehensively evaluating the health condition inside the user's mouth when analyzing captured images. For example, evaluation items may include the number of cavities, degree of progression of periodontal disease, and condition of the tooth surface. In addition, the analysis unit can compare the user's current health score with past scores to grasp changes in the health condition inside the mouth. Furthermore, the analysis unit can propose specific improvement measures and preventive measures to the user based on the health score. As a result, the user can comprehensively understand the health condition inside their mouth and take appropriate measures. Specifically, the analysis unit inputs high-resolution intraoral images (e.g., 3×1080×1920 RGB tensors) acquired by the camera unit into an image analysis AI model (e.g., CNN-based abnormality detection network) to automatically extract and quantify multiple evaluation items such as cavities, periodontal disease, tartar, pigmentation, and gum swelling. Examples of input to AI include “tooth surface image tensor” and “gum site image.” The analysis unit inputs these image features as health indicator vectors (e.g., number of cavities: 2, periodontal disease progression: 0.6, tartar amount: 0.3, etc.) into a scoring AI model (multilayer perceptron, decision tree, regression model) to calculate a comprehensive health score (0-100 points), site-specific scores, and risk classification (low, medium, high). Examples of output include “comprehensive score: 85 points,” “gum site score: 70 points,” and “risk classification: medium.” The analysis unit compares these scores with the user's past scores in a time series to automatically determine trends in health improvement or deterioration. Furthermore, based on score changes, the analysis unit automatically generates specific improvement measures and preventive measures such as “increase brushing frequency,” “recommend regular checkups,” and “recommend cleaning of specific areas,” and presents them in an easy-to-understand manner on the user interface. For AI model training, actual diagnostic results and treatment effect data are used as training data, and regression loss and classification loss are minimized. As a technical effect, the analysis unit can automate objective and quantitative health evaluation integrating multiple indicators and personalized improvement proposals, compared to conventional subjective and single-item evaluation, and strongly support user health management, preventive actions, and treatment planning. Application fields include self-care applications, dental diagnostic support, telemedicine, personalized healthcare services, and health progress monitoring.

The specifying unit can take into account the user's lifestyle habits and dietary tendencies when identifying areas requiring treatment based on analyzed results. For example, if the user frequently consumes sweets, the specifying unit focuses on identifying areas with a high risk of cavities. In addition, if the user is a smoker, the specifying unit can identify areas with a high risk of periodontal disease. Furthermore, the specifying unit can customize the identification results of areas requiring treatment based on the user's lifestyle habits and dietary tendencies. As a result, identification of areas requiring treatment tailored to the user's individual situation is possible, enabling more effective treatment. Specifically, the specifying unit integrates the list of abnormal site candidates output from the analysis unit (e.g., site-specific abnormal score vectors) and the user's lifestyle habit data (e.g., dietary history vectors, smoking/drinking habits, sleep duration, stress indicators), and inputs them into a risk-weighted AI model (e.g., regression model with abnormal score ×lifestyle risk weighting). Examples of input to AI include “abnormal score vector+frequency of sweet intake” and “gum abnormal score+smoking habit.” The specifying unit automatically generates risk profiles for each lifestyle habit from these input data and calculates the priority and treatment recommendation score for areas requiring treatment. The output from AI is obtained in formats such as “list of areas requiring treatment (with priority)” and “site-specific treatment recommendation score.” Examples of output include “upper right 6th: high cavity risk, treatment recommendation score: 0.92” and “lower left 3rd: medium periodontal disease risk, treatment recommendation score: 0.75.” Based on these outputs, the specifying unit automatically generates optimized treatment proposals and lifestyle improvement advice for each user and presents them on the user interface. For AI model training, lifestyle habits, dietary history, and treatment effect data are used as training data, and regression loss and classification loss are minimized. As a technical effect, the specifying unit can automate personalized treatment proposals that take into account individual risk factors, compared to conventional uniform identification of areas requiring treatment, and greatly improve treatment effect, preventive effect, and user satisfaction. Application fields include dental diagnostic support, self-care applications, personalized preventive medicine, and lifestyle disease countermeasure support.

The generation unit can create a 3D model of the user's oral cavity and display areas requiring treatment three-dimensionally when generating images of identified areas requiring treatment. For example, the generation unit generates a 3D model that reproduces details such as the surface irregularities of teeth and the condition of the gums. In addition, the generation unit can compare the identified areas requiring treatment with surrounding healthy areas and visually indicate the necessity of treatment. Furthermore, the generation unit can provide a function that allows the user to simulate the condition of the oral cavity after treatment. As a result, the user can confirm the effect of treatment in advance and deepen their understanding of the treatment. Specifically, the generation unit inputs intraoral images taken from multiple directions (e.g., 3×N×1080×1920 RGB tensors, where N is the number of shooting directions) and dental scan data (point cloud data, mesh data) into a 3D reconstruction AI model (e.g., 3D-CNN, VoxelNet, NeRF-based neural rendering model) to generate a 3D model of the oral cavity. Examples of input to AI include “multi-directional image tensor” and “dental point cloud data.” The generation unit accurately reproduces the surface shape of teeth, the three-dimensional structure of gums, and the spatial position of abnormal sites from these input data, and generates 3D visualization images that highlight and color-code areas requiring treatment. Furthermore, the generation unit can generate comparison images of healthy and abnormal areas and simulation images before and after treatment (e.g., tooth surface after whitening, gum shape after tartar removal). The output from AI is obtained in formats such as “3D mesh data,” “3D highlight image,” and “treatment simulation image.” Examples of output include “3D model with areas requiring treatment highlighted in red” and “simulation image after treatment.” Based on these outputs, the generation unit provides a user interface that allows the user to interactively rotate, zoom, and select areas on the 3D model, supporting pre-confirmation of treatment effects and promoting understanding of treatment content. For AI model training, actual 3D scan data and pre/post-treatment images are used as training data, and reconstruction loss and rendering loss are minimized. As a technical effect, the generation unit can automate three-dimensional and dynamic visualization of treatment areas and treatment effect simulation, compared to conventional 2D images and static explanations, and greatly improve user understanding, acceptance, and treatment action rate. Application fields include dental diagnostic support, self-care applications, telemedicine, treatment explanation support, and personalized healthcare services.

The sharing unit can estimate the user's emotions when sharing generated images, past symptoms, and treatment methods, and adjust the display method of shared information based on the estimated emotions. For example, when the user is relaxed, detailed information is displayed. In addition, when the user is tense, concise information can be displayed. Furthermore, when the user is in a hurry, information focusing on key points can be displayed. As a result, appropriate information can be provided according to the user's emotions, promoting user understanding. Specifically, the sharing unit acquires the user's facial images (3×224×224 RGB tensors) and voice spectrograms (1×128×256 2D arrays) in real time, and inputs these into a multimodal emotion estimation AI model (combination of convolutional neural network and recurrent neural network, or Transformer-based network). Examples of input to AI include “smiling facial image+calm voice spectrogram” and “frowning face+high-pitched tense voice.” The sharing unit extracts facial features and acoustic features from these input data and performs emotion classification with the AI model. The output from AI is obtained as emotion labels such as “relaxed,” “tense,” and “in a hurry” (output as softmax probability distribution for each emotion, e.g., relaxed: 0.82, tense: 0.12, in a hurry: 0.06) and emotion intensity scores (continuous values from 0.0 to 1.0). Examples of output include “relaxation level: 0.85” and “tension level: 0.65.” Based on these emotion estimation results, the shared information display control module executes rule-based control such as “if relaxation level is 0.7 or higher, display detailed shared information,” “if tension level is 0.5 or higher, display only concise shared information,” and “if in a hurry level is 0.5 or higher, display only key points.” Furthermore, the user's past emotional transitions and shared information viewing history are accumulated in a time-series database, and individually optimized display control logic (automatic learning of optimal information amount and display format for each user) can also be implemented. For AI model training, facial images and voice datasets labeled with emotions are used, and cross-entropy loss and data augmentation (facial expression changes, voice noise addition) are utilized to improve accuracy. As a technical effect, the sharing unit can automate flexible information presentation according to the user's psychological state, compared to conventional fixed information display and manual user selection, and greatly improve user understanding, satisfaction, and behavioral change rate. Application fields include self-care applications, dental diagnostic support, telemedicine, stress-free presentation of diagnostic results for children and the elderly, and personalized healthcare services.

The analysis unit can convert oral data of multiple patients into big data and, when generative AI analyzes it, estimate the user's emotions and adjust the display method of analysis results based on the estimated emotions. For example, when the user is relaxed, detailed analysis results are displayed. In addition, when the user is tense, concise analysis results can be displayed. Furthermore, when the user is in a hurry, analysis results focusing on key points can be displayed. As a result, appropriate information can be provided according to the user's emotions, promoting user understanding. Specifically, the analysis unit accumulates patient oral image data on the scale of tens of thousands to hundreds of millions of cases (e.g., 3×1080×1920 RGB tensors), diagnostic labels, treatment history, and lifestyle data in a distributed database, and uses a big data analysis AI model (e.g., distributed Transformer, graph neural network, clustering algorithm) to extract common symptoms, treatment patterns, and risk factors. Examples of input to AI include “distribution of cavity occurrence sites for all patients” and “treatment history+lifestyle habit vector.” Based on these big data analysis results, the analysis unit uses a user-specific emotion estimation AI (facial image and voice spectrogram input, CNN+RNN or Transformer) to estimate emotion labels and intensity scores, and the analysis result display control module executes rule-based control such as “display detailed statistical graphs and treatment trends when relaxed,” “display only summary of key points when tense,” and “display only important warnings when in a hurry.” Furthermore, the user's past emotional transitions and analysis result viewing history are accumulated in a time-series database, and individually optimized display control logic can also be implemented. For AI model training, facial images and voice datasets labeled with emotions and case datasets for big data analysis are used, and cross-entropy loss and clustering loss are utilized to improve accuracy. As a technical effect, the analysis unit can automate personalized information presentation and user psychology-adaptive flexible analysis result display utilizing vast patient data, compared to conventional simple statistical display and manual data analysis, and greatly improve user understanding, satisfaction, and treatment action rate. Application fields include medical big data analysis, self-care applications, dental diagnostic support, personalized preventive medicine, and telemedicine.

The camera unit can add an autofocus function for preferentially capturing specific areas inside the mouth during image capture. For example, the function may include automatically focusing on areas suspected of cavities. In addition, the camera unit can adjust the focus to capture the condition of the gums in detail. Furthermore, the camera unit can optimize the focus to detect fine scratches on the tooth surface. By adding an autofocus function, specific areas can be captured in detail. Specifically, the camera unit uses a real-time image analysis AI model (e.g., YOLO-based object detection network, segmentation CNN) to automatically detect regions such as suspected cavity areas, gums, and tooth surface scratches from captured images. Examples of input to AI include “intraoral image tensor” and “tooth surface close-up image.” The camera unit extracts coordinates and region masks of abnormal areas from these input data and outputs control signals such as “focus coordinates (x, y),” “zoom magnification,” and “depth of field” to the autofocus control module. Examples of output include “focus coordinates (320, 480), zoom 2.0x” and “focus coordinates (100, 900), zoom 1.5x.” Based on these outputs, the camera unit controls the lens drive motor in real time to automatically adjust the optimal focus, zoom, and exposure settings for specific areas. Furthermore, the camera unit can learn the history of abnormal areas and shooting tendencies for each user and implement individually optimized focus control logic. As a technical effect, the camera unit can greatly improve high-definition imaging of abnormal areas, diagnostic accuracy, and image data quality, compared to conventional manual focus and overall imaging. Application fields include dental diagnostic support, self-care applications, telemedicine, and intraoral abnormal area monitoring.

The camera unit can simultaneously measure the temperature and humidity inside the mouth during image capture and record them as additional information in the image. For example, the camera unit measures the temperature inside the mouth during image capture and records it in the image. In addition, the camera unit can measure the humidity inside the mouth during image capture and record it in the image. Furthermore, by combining temperature and humidity data, the camera unit can provide detailed information about the oral environment. By recording temperature and humidity information, detailed information about the oral environment can be provided. Specifically, the camera unit uses a high-precision temperature sensor (e.g., thermistor, infrared temperature sensor) and humidity sensor (e.g., capacitive humidity sensor) built into the toothbrush to acquire oral temperature (e.g., in 0.1° C. units) and humidity (e.g., in 1% units) in real time during image capture. Examples of acquired data include “temperature: 36.8°C.” and “humidity: 85%.” The camera unit links these sensor data with image data and timestamps and saves them as image metadata. Furthermore, the camera unit can input temperature and humidity data into the analysis unit or AI model to detect abnormalities in the oral environment (e.g., risk assessment of bacterial proliferation due to high temperature and humidity) and calculate health scores. Examples of input to AI include “image tensor+temperature: 36.8° C.+humidity: 85%.” The output from AI is obtained in formats such as “environment abnormality label” and “risk score.” Examples of output include “high humidity risk: 0.78” and “within normal range.” Based on these outputs, the camera unit can automatically generate proposals for improving the oral environment (e.g., ventilation recommendation, water intake recommendation) for the user. As a technical effect, the camera unit can automate multifaceted health evaluation and abnormality detection that takes into account environmental factors such as temperature and humidity, compared to conventional diagnosis using only images, and greatly improve diagnostic accuracy, preventive effect, and user satisfaction. Application fields include dental diagnostic support, self-care applications, telemedicine, and oral environment monitoring.

The camera unit can estimate the user's emotions and determine the priority of areas to be captured based on the estimated emotions. For example, when the user is relaxed, overall oral imaging is prioritized. In addition, when the user is tense, imaging of specific areas can be prioritized. Furthermore, when the user is in a hurry, imaging of important areas can be prioritized. By determining the priority of areas to be captured according to the user's emotions, appropriate areas can be captured. Specifically, the camera unit acquires the user's facial images (3×224×224 RGB tensors) and voice spectrograms (1×128×256) in real time and inputs them into a multimodal emotion estimation AI model (CNN +RNN or Transformer). Examples of input to AI include “smiling face+calm voice” and “frowning face+high-pitched voice.” The camera unit estimates emotion labels and intensity scores (e.g., relaxed: 0.80, tense: 0.15, in a hurry: 0.05) from these input data, and the area capture priority determination module executes rule-based control such as “prioritize overall imaging when relaxed,” “prioritize close-up imaging of abnormal areas when tense,” and “prioritize imaging of only areas requiring treatment when in a hurry.” Furthermore, the user's past emotional transitions and imaging history are accumulated in a time-series database, and individually optimized area selection logic can also be implemented. For AI model training, facial images and voice datasets labeled with emotions are used, and cross-entropy loss and data augmentation are utilized to improve accuracy. As a technical effect, the camera unit can automate flexible area selection for imaging according to the user's psychological state, compared to conventional fixed imaging order and manual selection, and greatly improve user convenience, satisfaction, and diagnostic accuracy. Application fields include self-care applications, dental diagnostic support, telemedicine, stress-free presentation of diagnostic results, and personalized healthcare services.

The analysis unit can analyze the bacterial balance inside the mouth during analysis and detect abnormal balance. For example, the analysis unit analyzes the bacterial balance inside the mouth and detects abnormal balance. In addition, the analysis unit can propose appropriate treatment methods based on the results of bacterial balance analysis. Furthermore, the analysis unit can detect abnormal bacterial balance at an early stage and prompt treatment. By analyzing bacterial balance, abnormal balance can be detected early and treatment can be prompted. Specifically, the analysis unit integrates bacterial DNA sequence data obtained from the oral cavity (16S rRNA sequence data, thousands to tens of thousands of base sequences), presence ratio vectors for each bacterial species (30-dimensional bacterial species ratio vector), and image features (such as degree of gum swelling and amount of tartar), and inputs them into a multimodal AI model (neural network+clustering algorithm). Examples of input to AI include “bacterial species ratio vector+gum image features” and “16S sequence data+tartar amount.” The analysis unit uses these input data to extract diversity indices of bacterial species and increase/decrease patterns of specific bacterial species, and performs normal/abnormal judgment and risk score calculation with the AI model. The output from AI is obtained as labels and scores such as “normal,” “abnormal (balance collapse),” and “risk score: 0.78.” Examples of output include “bacterial balance abnormality detected: risk 0.82” and “within normal range.” Based on these outputs, the analysis unit links abnormal balance detection results to the treatment proposal module and automatically proposes treatment methods such as “probiotics recommended” and “antibacterial treatment recommended.” Furthermore, the analysis unit analyzes time-series changes in bacterial balance to realize early detection of abnormal trends and monitoring of treatment effects. For AI model training, datasets labeled with bacterial species and treatment effect data are used, and cross-entropy loss and clustering loss are utilized. As a technical effect, the analysis unit can automatically analyze vast bacterial species data and realize early detection of abnormal balance, treatment proposals, and progress monitoring with high accuracy and efficiency, compared to conventional culture methods and subjective evaluation by humans. Application fields include dental diagnostic support, self-care applications, telemedicine, oral bacterial monitoring, and medical big data analysis.

Below, the processing flow of Example of the Embodiment is briefly described. Specifically, the system operates in cooperation among modules such as acquisition of high-definition images and various sensor data by the camera unit, AI image analysis and health score calculation by the analysis unit, personalized identification of areas requiring treatment by the specifying unit, 3D model generation and treatment simulation by the generation unit, information sharing with authority management by the sharing unit, and big data analysis and algorithm optimization by the analysis unit. By describing in detail examples of AI input/output at each step, data flow, subsequent processing, technical effects, and application fields, the improvement story of computer technology is clarified and differentiation from conventional technology is achieved.

Step 1: The camera unit captures images of the inside of the mouth. For example, the camera unit captures detailed images of the inside of the mouth using a camera built into a toothbrush. The camera unit can capture high-resolution images of, for example, the surface of teeth and the condition of gums. Step 2: The analysis unit analyzes images captured by the camera unit using AI. For example, the analysis unit detects signs of cavities or periodontal disease based on images captured by AI. The analysis unit analyzes images using deep learning or machine learning algorithms. Step 3: The specifying unit identifies areas requiring treatment based on results analyzed by the analysis unit. For example, the specifying unit identifies areas requiring treatment for cavities or periodontal disease based on the analysis results. Step 4: The generation unit generates images of the areas requiring treatment identified by the specifying unit. For example, the generation unit generates 2D images or 3D models of the identified areas requiring treatment. Step 5: The sharing unit shares images generated by the generation unit or past symptoms and treatment methods between a patient and a doctor. For example, the sharing unit shares information through a healthcare application. When the patient opens the application, images of areas requiring treatment, past symptoms, and treatment methods are displayed. Step 6: The analysis unit converts oral data of tens of thousands to hundreds of millions of patients into big data and generative AI analyzes it. For example, the analysis unit analyzes common symptoms and treatment methods based on the collected data and proposes new treatment methods and preventive measures. Specifically, in Step 1, the camera unit simultaneously acquires 3×1080×1920 RGB image tensors and sensor data such as temperature, humidity, odor, and sound, and records them as image metadata. In Step 2, the analysis unit inputs these image and sensor data into CNN or Transformer-based AI models to automatically detect abnormal areas such as cavities, periodontal disease, tartar, pigmentation, and swelling, and calculates health indicator vectors. Examples of AI input are “tooth surface image tensor” and “gum image+temperature 36.5° C.+humidity 85%.” AI output includes “abnormal area label” and “health score: 85 points.” In Step 3, the specifying unit integrates analysis results and user lifestyle habit data and uses a risk-weighted AI model to calculate the priority and treatment recommendation score for areas requiring treatment. Examples of AI input are “abnormal score vector+frequency of sweet intake,” and AI output is “list of areas requiring treatment (with priority).” In Step 4, the generation unit inputs multi-directional images and dental point cloud data into a 3D reconstruction AI model to generate 3D models highlighting areas requiring treatment and treatment simulation images. Examples of AI input are “multi-directional image tensor” and “dental point cloud data,” and AI output is “3D mesh data” and “treatment simulation image.” In Step 5, the sharing unit uses a user emotion estimation AI (facial image and voice spectrogram input) to estimate emotion labels and intensity scores, and the display control module executes rule-based control such as “display detailed information when relaxed,” “display concise information when tense,” and “display only key points when in a hurry.” In Step 6, the analysis unit uses a big data analysis AI model to extract common symptoms, treatment patterns, and risk factors from all patient data and automatically generates personalized treatment proposals and preventive measures for each user. AI models at each step are trained using cross-entropy loss, regression loss, and clustering loss, and accuracy is continuously improved through online learning and transfer learning. As a technical effect, the system can realize automated, personalized diagnosis, treatment proposals, and progress monitoring utilizing vast data with high accuracy and efficiency, compared to conventional manual and static diagnosis, and can be applied to various fields such as dental diagnostic support, self-care, telemedicine, medical big data analysis, and personalized healthcare services.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the above-mentioned elements, including the camera unit, analysis unit, specifying unit, generation unit, sharing unit, and analysis unit, is implemented by at least one of, for example, a smart device 14 and a data processing apparatus 12. For example, the camera unit is implemented by a camera 42 of the smart device 14 and captures detailed images of the inside of the mouth. The analysis unit is implemented, for example, by a specific processing unit 290 of the data processing apparatus 12 and analyzes images captured using AI. The specifying unit is implemented, for example, by the specific processing unit 290 of the data processing apparatus 12 and identifies areas requiring treatment based on the analysis results. The generation unit is implemented, for example, by the specific processing unit 290 of the data processing apparatus 12 and generates images of the identified areas requiring treatment. The sharing unit is implemented, for example, by a control unit 46A of the smart device 14 and shares generated images, past symptoms, and treatment methods between a patient and a doctor. The analysis unit is implemented, for example, by the specific processing unit 290 of the data processing apparatus 12 and converts oral data of tens of thousands to hundreds of millions of patients into big data and analyzes it using generative AI. The correspondence between each unit and the apparatus or control unit is not limited to the examples described above and various modifications are possible.

### Second Embodiment

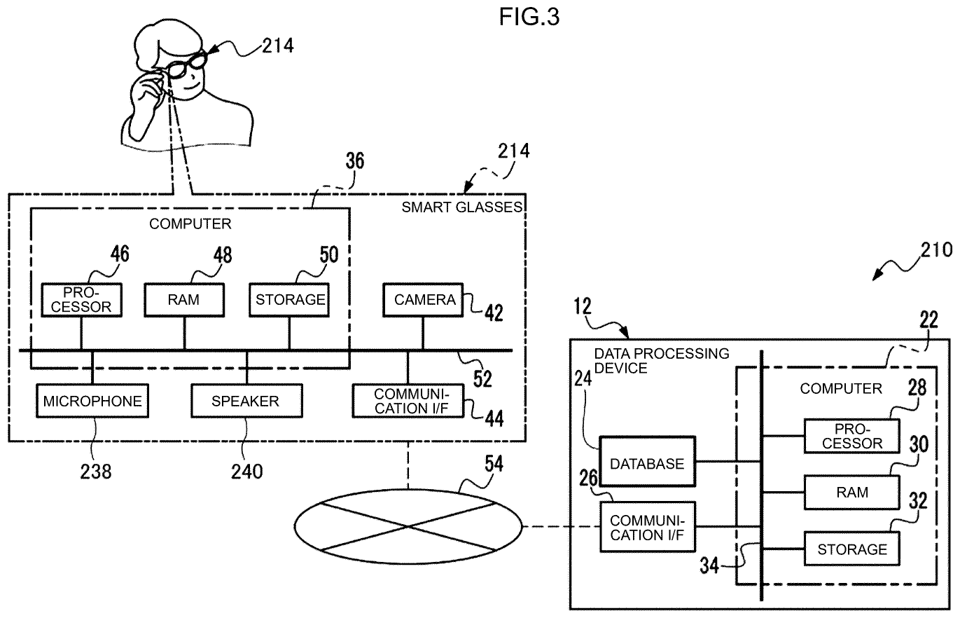

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

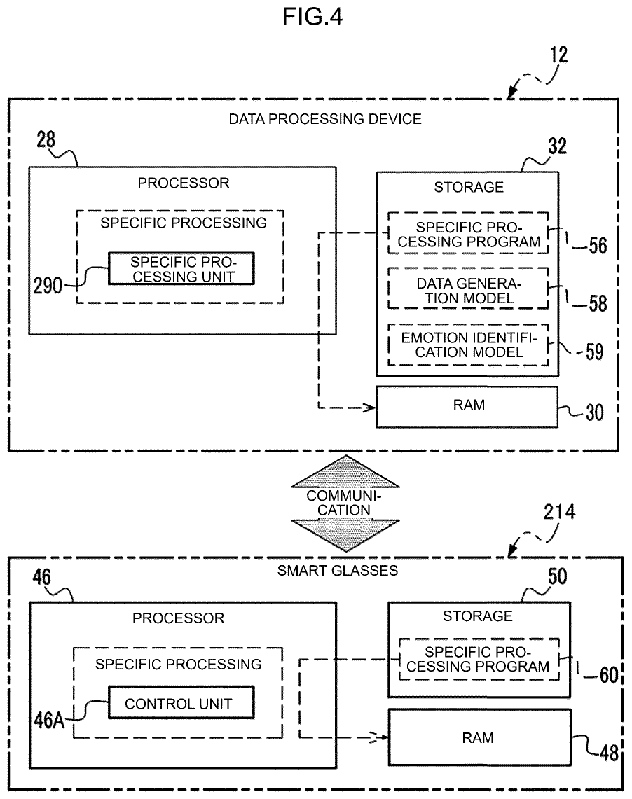

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the above-mentioned elements, including the camera unit, analysis unit, specifying unit, generation unit, sharing unit, and analysis unit, is implemented by at least one of, for example, smart glasses 214 and a data processing apparatus 12. For example, the camera unit is implemented by a camera 42 of the smart glasses 214 and captures detailed images of the inside of the mouth. The analysis unit is implemented, for example, by a specific processing unit 290 of the data processing apparatus 12 and analyzes images captured using AI. The specifying unit is implemented, for example, by the specific processing unit 290 of the data processing apparatus 12 and identifies areas requiring treatment based on the analysis results. The generation unit is implemented, for example, by the specific processing unit 290 of the data processing apparatus 12 and generates images of the identified areas requiring treatment. The sharing unit is implemented, for example, by a control unit 46A of the smart glasses 214 and shares generated images, past symptoms, and treatment methods between a patient and a doctor. The analysis unit is implemented, for example, by the specific processing unit 290 of the data processing apparatus 12 and converts oral data of tens of thousands to hundreds of millions of patients into big data and analyzes it using generative AI. The correspondence between each unit and the apparatus or control unit is not limited to the examples described above and various modifications are possible.

### Third Embodiment

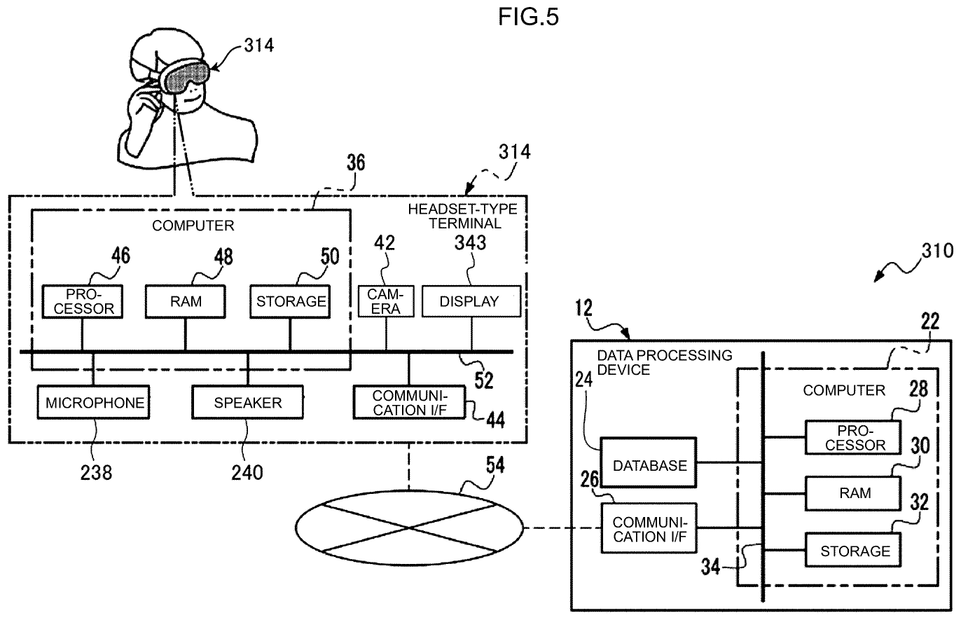

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

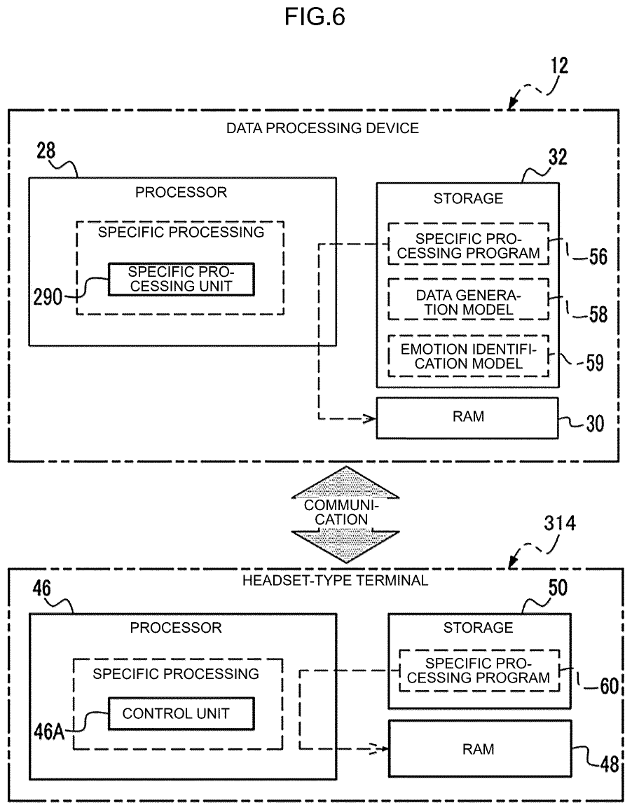

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the above-mentioned elements, including the camera unit, analysis unit, specifying unit, generation unit, sharing unit, and analysis unit, is implemented by at least one of, for example, a headset-type terminal 314 and a data processing apparatus 12. For example, the camera unit is implemented by a camera 42 of the headset-type terminal 314 and captures detailed images of the inside of the mouth. The analysis unit is implemented, for example, by a specific processing unit 290 of the data processing apparatus 12 and analyzes images captured using AI. The specifying unit is implemented, for example, by the specific processing unit 290 of the data processing apparatus 12 and identifies areas requiring treatment based on the analysis results. The generation unit is implemented, for example, by the specific processing unit 290 of the data processing apparatus 12 and generates images of the identified areas requiring treatment. The sharing unit is implemented, for example, by a control unit 46A of the headset-type terminal 314 and shares generated images, past symptoms, and treatment methods between a patient and a doctor. The analysis unit is implemented, for example, by the specific processing unit 290 of the data processing apparatus 12 and converts oral data of tens of thousands to hundreds of millions of patients into big data and analyzes it using generative AI. The correspondence between each unit and the apparatus or control unit is not limited to the examples described above and various modifications are possible.

### Fourth Embodiment

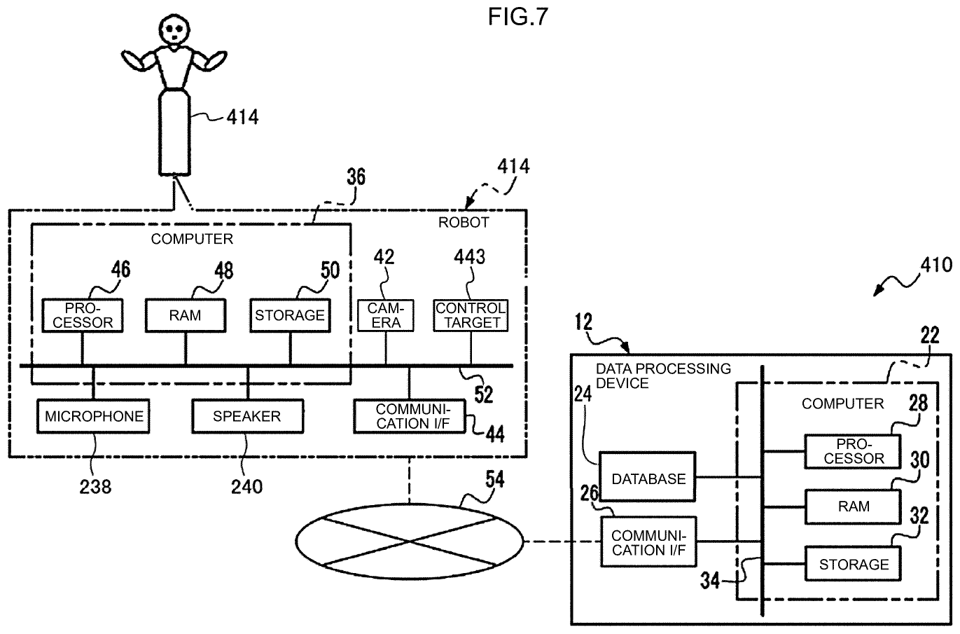

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

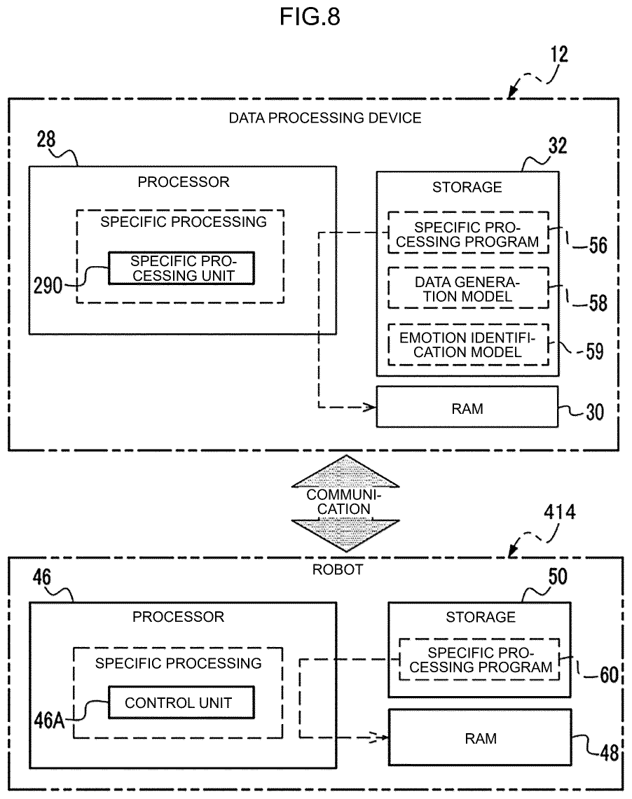

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the above-mentioned elements, including the camera unit, analysis unit, specifying unit, generation unit, sharing unit, and analysis unit, is implemented by at least one of, for example, a robot 414 and a data processing apparatus 12. For example, the camera unit is implemented by a camera 42 of the robot 414 and captures detailed images of the inside of the mouth. The analysis unit is implemented, for example, by a specific processing unit 290 of the data processing apparatus 12 and analyzes images captured using AI. The specifying unit is implemented, for example, by the specific processing unit 290 of the data processing apparatus 12 and identifies areas requiring treatment based on the analysis results. The generation unit is implemented, for example, by the specific processing unit 290 of the data processing apparatus 12 and generates images of the identified areas requiring treatment. The sharing unit is implemented, for example, by a control unit 46A of the robot 414 and shares generated images, past symptoms, and treatment methods between a patient and a doctor. The analysis unit is implemented, for example, by the specific processing unit 290 of the data processing apparatus 12 and converts oral data of tens of thousands to hundreds of millions of patients into big data and analyzes it using generative AI. The correspondence between each unit and the apparatus or control unit is not limited to the examples described above and various modifications are possible.

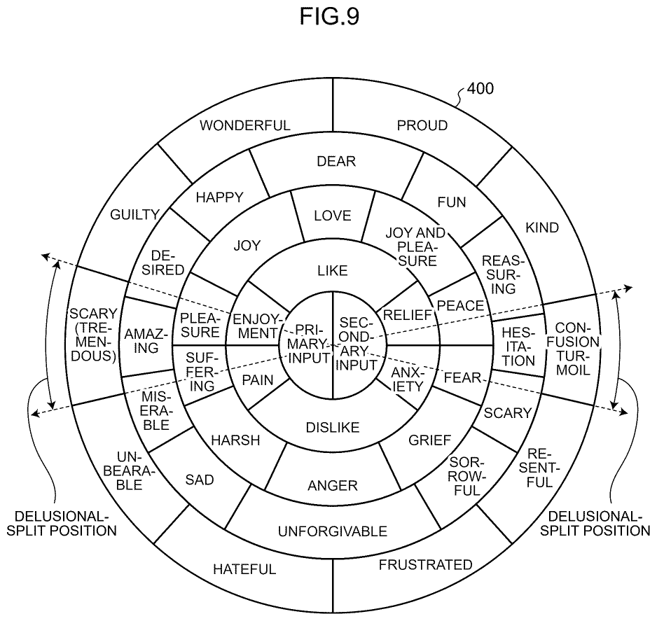

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

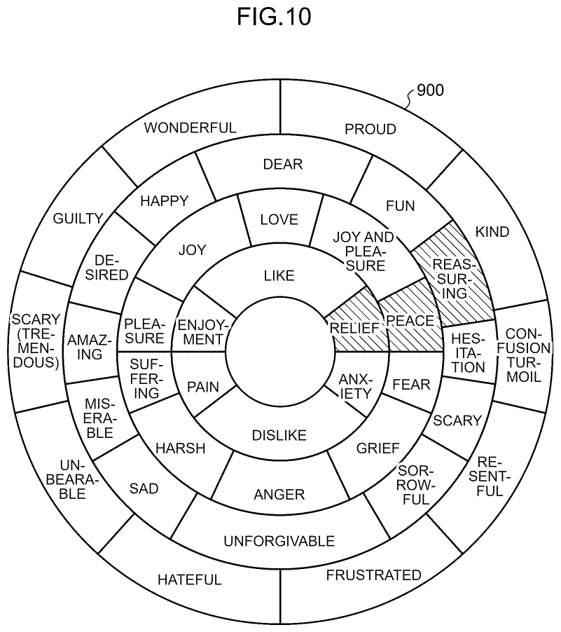

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.

### Supplementary Note 1

A system comprising: a camera unit configured to capture images of the inside of the mouth; an analysis unit configured to analyze images captured by the camera unit using AI; a specifying unit configured to identify areas requiring treatment based on results analyzed by the analysis unit; a generation unit configured to generate images of the areas requiring treatment identified by the specifying unit; a sharing unit configured to share images generated by the generation unit or past symptoms and treatment methods between a patient and a doctor; and an analysis unit configured to convert oral data of a plurality of patients into big data and analyze it using generative AI.

### Supplementary Note 2

The system according to Supplementary Note 1, wherein the camera unit captures detailed images of the inside of the mouth using a camera built into a toothbrush.

### Supplementary Note 3

The system according to Supplementary Note 1, wherein the analysis unit analyzes images captured by AI and detects signs of cavities or periodontal disease.

### Supplementary Note 4

The system according to Supplementary Note 1, wherein the specifying unit identifies areas requiring treatment based on the analyzed results.

### Supplementary Note 5

The system according to Supplementary Note 1, wherein the generation unit generates images of the identified areas requiring treatment.

### Supplementary Note 6

The system according to Supplementary Note 1, wherein the sharing unit shares generated images or past symptoms and treatment methods between a patient and a doctor through a healthcare application.

### Supplementary Note 7

The system according to Supplementary Note 1, wherein the analysis unit converts oral data of a plurality of patients into big data and analyzes it using generative AI.

### Supplementary Note 8

The system according to Supplementary Note 1, wherein the camera unit estimates the user's emotions and adjusts the timing of image capture based on the estimated emotions.

### Supplementary Note 9

The system according to Supplementary Note 1, wherein the camera unit adds an autofocus function for preferentially capturing specific areas inside the mouth during image capture.

### Supplementary Note 10

The system according to Supplementary Note 1, wherein the camera unit simultaneously measures the temperature and humidity inside the mouth during image capture and records them as additional information in the image.

### Supplementary Note 11

The system according to Supplementary Note 1, wherein the camera unit estimates the user's emotions and determines the priority of areas to be captured based on the estimated emotions.

### Supplementary Note 12

The system according to Supplementary Note 1, wherein the camera unit simultaneously collects audio data inside the mouth during image capture and detects abnormal sounds. (Supplementary Note 13

The system according to Supplementary Note 1, wherein the camera unit is equipped with an odor sensor inside the mouth during image capture and detects abnormal odors.

### Supplementary Note 14

The system according to Supplementary Note 1, wherein the analysis unit estimates the user's emotions and adjusts the display method of analysis results based on the estimated emotions.

### Supplementary Note 15

The system according to Supplementary Note 1, wherein the analysis unit optimizes the analysis algorithm by referring to past analysis data during analysis.

### Supplementary Note 16

The system according to Supplementary Note 1, wherein the analysis unit introduces a scoring system for comprehensively evaluating the health condition inside the mouth during analysis.

### Supplementary Note 17

The system according to Supplementary Note 1, wherein the analysis unit estimates the user's emotions and determines the priority of analysis results based on the estimated emotions.

### Supplementary Note 18

The system according to Supplementary Note 1, wherein the analysis unit analyzes the bacterial balance inside the mouth during analysis and detects abnormal balance.

### Supplementary Note 19

The system according to Supplementary Note 1, wherein the analysis unit analyzes pigmentation inside the mouth during analysis and detects abnormal pigmentation.

### Supplementary Note 20

The system according to Supplementary Note 1, wherein the specifying unit estimates the user's emotions and adjusts the display method of identification results based on the estimated emotions.

### Supplementary Note 21

The system according to Supplementary Note 1, wherein the specifying unit optimizes the identification algorithm by referring to past identification data during identification.

### Supplementary Note 22

The system according to Supplementary Note 1, wherein the specifying unit introduces a scoring system for comprehensively evaluating the health condition inside the mouth during identification.

### Supplementary Note 23

The system according to Supplementary Note 1, wherein the specifying unit estimates the user's emotions and determines the priority of identification results based on the estimated emotions.

### Supplementary Note 24

The system according to Supplementary Note 1, wherein the specifying unit identifies the bacterial balance inside the mouth during identification and detects abnormal balance.

### Supplementary Note 25

The system according to Supplementary Note 1, wherein the specifying unit identifies pigmentation inside the mouth during identification and detects abnormal pigmentation. (Supplementary Note 26

The system according to Supplementary Note 1, wherein the generation unit estimates the user's emotions and adjusts the display method of generated images based on the estimated emotions.

### Supplementary Note 27

The system according to Supplementary Note 1, wherein the generation unit optimizes the generation algorithm by referring to past generation data during generation.

### Supplementary Note 28

The system according to Supplementary Note 1, wherein the generation unit introduces a scoring system for comprehensively evaluating the health condition inside the mouth during generation.

### Supplementary Note 29

The system according to Supplementary Note 1, wherein the generation unit estimates the user's emotions and determines the priority of generated images based on the estimated emotions.

### Supplementary Note 30

The system according to Supplementary Note 1, wherein the generation unit generates the bacterial balance inside the mouth during generation and detects abnormal balance.

### Supplementary Note 31

The system according to Supplementary Note 1, wherein the generation unit generates pigmentation inside the mouth during generation and detects abnormal pigmentation.

### Supplementary Note 32

The system according to Supplementary Note 1, wherein the sharing unit estimates the user's emotions and adjusts the display method of shared information based on the estimated emotions.

### Supplementary Note 33

The system according to Supplementary Note 1, wherein the sharing unit optimizes the sharing algorithm by referring to past sharing data during sharing. (Supplementary Note 34

The system according to Supplementary Note 1, wherein the sharing unit introduces a scoring system for comprehensively evaluating the health condition inside the mouth during sharing.

### Supplementary Note 35

The system according to Supplementary Note 1, wherein the sharing unit estimates the user's emotions and determines the priority of shared information based on the estimated emotions.

### Supplementary Note 36

The system according to Supplementary Note 1, wherein the sharing unit shares the bacterial balance inside the mouth during sharing and detects abnormal balance.

### Supplementary Note 37

The system according to Supplementary Note 1, wherein the sharing unit shares pigmentation inside the mouth during sharing and detects abnormal pigmentation.

### Supplementary Note 38

The system according to Supplementary Note 1, wherein the analysis unit estimates the user's emotions and adjusts the display method of analysis results based on the estimated emotions.

### Supplementary Note 39

The system according to Supplementary Note 1, wherein the analysis unit optimizes the analysis algorithm by referring to past analysis data during analysis.

### Supplementary Note 40

The system according to Supplementary Note 1, wherein the analysis unit introduces a scoring system for comprehensively evaluating the health condition inside the mouth during analysis.

### Supplementary Note 41

The system according to Supplementary Note 1, wherein the analysis unit estimates the user's emotions and determines the priority of analysis results based on the estimated emotions.

### Supplementary Note 42

The system according to Supplementary Note 1, wherein the analysis unit analyzes the bacterial balance inside the mouth during analysis and detects abnormal balance.

### Supplementary Note 43

The system according to Supplementary Note 1, wherein the analysis unit analyzes pigmentation inside the mouth during analysis and detects abnormal pigmentation.

What is claimed is:

1. A system comprising:
circuitry configured to:
receive, via a packet-switched network, image data captured by a sensor of a client terminal;
apply a convolutional neural network to the image data to generate a segmentation map indicating classification labels for regions of the image data;
identify, based on the segmentation map, one or more target regions having a classification score exceeding a threshold;
generate, by inputting feature vectors extracted from the one or more target regions into a data generation model, rendered image data representing the one or more target regions;
transmit, via the packet-switched network, the rendered image data and record data associated with the one or more target regions to the client terminal; and
aggregate stored data sets received from a plurality of client terminals into a distributed database and apply a generative model to the aggregated stored data sets to generate inference data comprising pattern labels and prediction scores.

2. The system according to claim 1, wherein the image data comprises intraoral image data captured by a camera integrated into a handheld device, and wherein the intraoral image data represents at least one of a surface condition of a tooth or a condition of gingival tissue.

3. The system according to claim 1, wherein the convolutional neural network comprises a Vision Transformer, and wherein the segmentation map comprises an abnormal probability map assigning a continuous value from 0.0 to 1.0 to each pixel of the image data.

4. The system according to claim 1, wherein identifying the one or more target regions comprises applying rule-based logic to assign a priority to each of the one or more target regions when a plurality of target regions is detected simultaneously.

5. The system according to claim 1, wherein generating the rendered image data comprises applying a three-dimensional reconstruction algorithm to multi-view image data of the one or more target regions to generate a three-dimensional mesh model.

6. The system according to claim 1, wherein the circuitry is further configured to transmit the rendered image data and the record data to the client terminal using an encrypted communication protocol, and wherein the record data comprises time-series data indicating past conditions and past actions associated with the one or more target regions.

7. The system according to claim 1, wherein the generative model comprises a large language model, and wherein the inference data further comprises at least one of a proposed action text, a risk factor ranking, or a cluster label.

8. The system according to claim 1, wherein the circuitry is further configured to estimate an emotion of a user by applying an emotion identification model to at least one of facial image data or voice spectrogram data received from the client terminal, and to adjust a timing of receiving the image data based on the estimated emotion.

9. The system according to claim 1, wherein the circuitry is further configured to detect, from the image data, a candidate region using an object detection network, generate a focus control signal comprising a recommended focus score for the candidate region, and transmit the focus control signal to the client terminal to cause the sensor to adjust autofocus to the candidate region.

10. The system according to claim 1, wherein the circuitry is further configured to receive, via the packet-switched network, environmental sensor data comprising at least one of temperature data or humidity data captured concurrently with the image data, and to record the environmental sensor data as metadata associated with the image data.

11. The system according to claim 10, wherein the circuitry is further configured to apply an anomaly detection neural network to the environmental sensor data and the image data to generate a risk score, and to transmit the risk score to the client terminal.

12. The system according to claim 1, wherein the circuitry is further configured to estimate an emotion of a user by applying an emotion identification model to sensor data received from the client terminal, and to determine a priority of the one or more target regions based on the estimated emotion, such that when the estimated emotion indicates relaxation, the circuitry prioritizes comprehensive identification, and when the estimated emotion indicates urgency, the circuitry prioritizes identification of a subset of the one or more target regions.

13. The system according to claim 1, wherein the circuitry is further configured to compute a composite score by inputting a multidimensional vector comprising a plurality of evaluation items derived from the segmentation map into a scoring model, the composite score indicating an overall condition assessment on a numerical scale.

14. The system according to claim 1, wherein the circuitry is further configured to analyze the image data to detect a distribution pattern of biological material and to generate a balance assessment label indicating whether the distribution pattern is normal or abnormal.

15. The system according to claim 1, wherein the circuitry is further configured to analyze the image data to detect a pigmentation pattern and to generate a pigmentation assessment label indicating whether the pigmentation pattern is normal or abnormal.

16. The system according to claim 1, wherein the circuitry is further configured to acquire past analysis data from the distributed database, and to optimize at least one of a weight parameter, a hyperparameter, or an algorithm selection of the convolutional neural network based on the past analysis data using at least one of online learning or transfer learning.

17. The system according to claim 1, wherein the circuitry is further configured to estimate an emotion of a user by applying an emotion identification model to at least one of a facial image tensor or a voice spectrogram received from the client terminal, and to adjust a display format of the rendered image data based on the estimated emotion, such that when the estimated emotion indicates relaxation, the circuitry generates detailed rendered image data, and when the estimated emotion indicates urgency, the circuitry generates summarized rendered image data.

18. A system comprising:
circuitry configured to:
receive, via a packet-switched network, an RGB image tensor captured by a CMOS sensor of a client terminal, the RGB image tensor having dimensions corresponding to three color channels and a resolution of at least 1920 by 1080 pixels;
apply a convolutional neural network comprising a Vision Transformer to the RGB image tensor to generate a segmentation map comprising an abnormal probability map assigning a continuous classification value from 0.0 to 1.0 to each pixel of the RGB image tensor;
identify, based on the segmentation map, one or more target regions having a classification value exceeding a threshold of 0.7, and generate a priority list of the one or more target regions by applying rule-based logic;
generate, by inputting feature vectors extracted from the one or more target regions into a data generation model obtained by deep learning on a neural network, rendered image data comprising at least one of a two-dimensional image with highlighted target regions or a three-dimensional mesh model generated by a multi-view stereo reconstruction algorithm;
transmit, via the packet-switched network using an encrypted communication protocol, the rendered image data and time-series record data associated with the one or more target regions to the client terminal; and
aggregate stored data sets comprising image data, record data, and attribute vectors received from a plurality of client terminals into a distributed database, and apply a generative model comprising a large language model to the aggregated stored data sets to generate inference data comprising at least one of a prediction score, a proposed action text, or a risk factor ranking.

19. The system according to claim 18, wherein the data generation model comprises at least one of a text generation AI, an image generation AI, or a multimodal generation AI, and wherein the data generation model is a fine-tuned model configured to output inference results from prompts without instructions.

20. A method performed by circuitry of a system, the method comprising:
receiving, via a packet-switched network, image data captured by a sensor of a client terminal;
applying a convolutional neural network to the image data to generate a segmentation map indicating classification labels for regions of the image data;
identifying, based on the segmentation map, one or more target regions having a classification score exceeding a threshold;
generating, by inputting feature vectors extracted from the one or more target regions into a data generation model, rendered image data representing the one or more target regions;
transmitting, via the packet-switched network, the rendered image data and record data associated with the one or more target regions to the client terminal; and
aggregating stored data sets received from a plurality of client terminals into a distributed database and applying a generative model to the aggregated stored data sets to generate inference data comprising pattern labels and prediction scores.
